(19) United States
(12) Patent Application Publication
(10) Pub. No.: US 2025/0109104 A1
(43) Pub. Date: Apr. 3, 2025

(54) SQUARAINE FLUOROPHORES

(71) Applicants: The General Hospital Corporation, Boston, MA (US); Georgia State University Research Foundation, Inc., Atlanta, GA (US)

(72) Inventors: Maged Henary, Lawrenceville, GA (US); Hak Soo Choi, Needham, MA (US); Satoshi Kashiwagi, Boston, MA (US)

(21) Appl. No.: 18/833,221

(22) PCT Filed: Feb. 8, 2023
(86) PCT No.: PCT/US2023/062230

§ 371 Date: Jul. 25, 2024

Related U.S. Application Data
(60) Provisional application No. 63/307,857, filed on Feb. 8, 2022.

(51) Int. Cl. C07D 209/24; A61K 49/00; C07D 209/20; C07D 209/26; C07D 403/14

(52) U.S. Cl. CPC C07D 209/24; A61K 49/0021; C07D 209/20; C07D 209/26; C07D 403/14

(57) ABSTRACT

The present invention is directed to squaraine fluorophores, methods of making squaraine fluorophores, and methods of using squaraine fluorophores. In some embodiments, squaraine fluorophores can be used as imaging agents, for example for imaging cancer. In some embodiments, the squaraine fluorophores can be used to guide surgical tumor resection.

### CLAIM OF PRIORITY

This application claims priority to U.S. Patent Application Ser. No. 63/307,857, filed on Feb. 8, 2022, the entire contents of which are hereby incorporated by reference.

### FEDERALLY SPONSORED RESEARCH OR DEVELOPMENT

This invention was made with Government support under Grant No. EB022230 awarded by the National Institutes of Health. The Government has certain rights in the invention.

### TECHNICAL FIELD

The present invention is directed to squaraine fluorophores, methods of making squaraine fluorophores, and methods of using squaraine fluorophores. In some embodiments, squaraine fluorophores can be used as imaging agents, for example for imaging cancer. In some embodiments, the squaraine fluorophores can be used to guide surgical tumor resection.

### BACKGROUND

There are numerous deadly diseases affecting current human population. For example, cancer is one of the leading causes of death in contemporary society. Currently, cancer incidence is nearly 450 cases of cancer per 100,000 men and women per year, while cancer mortality is nearly 71 cancer deaths per 100,000 men and women per year. The socioeconomic burden of cancer is substantial and reflects both healthcare spending as well as lost productivity due to co-morbidities and premature death. Healthcare spending on treating cancer exceed tens of billions of dollars worldwide. However, the economic burden of lost productivity due to cancer is over 60% of the total economic burden associated with cancer. Prevention, early detection, and effective treatment help reduce this economic burden.

### SUMMARY

The present disclosure provides squaraine chromophores for fast and efficient targeting of cancers and for fluorescence image-guided surgery. Advantageously, zwitterionic and planar structured squaraines within the present claims are water-soluble and stable in aqueous solutions due to the molecules' 3D conformation in which the cyclic “core” is protected, resulting in reduced serum binding and superb optical properties in the NIR wavelengths, allowing surgeons to more precisely perform surgical tumor resection. Currently, an FDA-approved small molecule NIR probe indocyanine green (ICG) is available in the clinic. However, ICG shows no specific cancer targeting capability and has significant limitations in image-guided cancer surgery due to the short blood half-life and nonspecific uptake. In addition, ICG is not suitable for cancer imaging since it does not have a targeting moiety in the backbone of chromophore. Hence, ICG cannot be expected to display on-target binding. A fluorescent molecule for cancer imaging must accumulate in cancer cells via active and/or passive targeting mechanisms and be stable for clinical use. Advantageously, the fluorophore compounds within the present claims allow for “structure-inherent targeting (SIT)” by tuning the lipophilicity and charge of the molecular backbone structures. The SIT strategy allows for high uptake in specific tissues (e.g., cancer) with low nonspecific binding.

In one general aspect, the present disclosure provides compound of Formula (II):

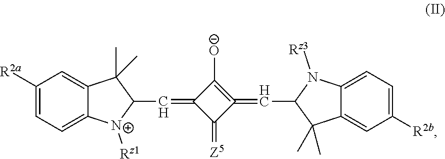

or a pharmaceutically acceptable salt thereof, wherein:
Z⁵ is O, S, NR^{a}, or CR^{a}R^{b};
R^{a} is F, Cl, Br, I, NO₂, CN, R^{a1}, OR^{a1}, SR^{a1}, N(R^{a1})₂, SO₂R^{a1}, SO₂N(R^{a1})₂, C(O)R^{a1}, C(O)OR^{a1}, OC(O)R^{a1}, C(O)N(R^{a1})₂, N(R^{a1})C(O)R^{a1}, OC(O)N(R^{a1})₂, or N(R^{a1})C(O)N(R^{a1})₂, wherein each R^{a1} is independently selected from hydrogen, C₁₋₈ alkyl, aryl, C₁₋₈ heteroaryl, C₃₋₈ cycloalkyl, and C₁₋₈ heterocyclyl; and
R^{b} is F, Cl, Br, I, NO₂, CN, R^{b1}, OR^{b1}, SR^{b1}, N(R^{b1})₂, SO₂R^{b1}, SO₂N(R^{b1})₂, C(O)R^{b1}, C(O)OR^{b1}, OC(O)R^{b1}, C(O)N(R^{b1})₂, N(R^{b1})C(O)R^{b1}, OC(O)N(R^{b1})₂, or N(R^{b1})C(O)N(R^{b1})₂, wherein each R^{b1} is independently selected from hydrogen, C₁₋₈ alkyl, aryl, C₁₋₈ heteroaryl, C₃₋₈ cycloalkyl, and C₁₋₈ heterocyclyl;
or R^{a} and R^{b} together form a ring;
R^{2a} is selected from F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, and N(R^{2a*})C(O)N(R^{2a*})₂, wherein R^{2a*} is in each case independently selected from hydrogen, C₁₋₈ alkyl, aryl, C₁₋₈ heteroaryl, C₃₋₈ cycloalkyl, and C₁₋₈ heterocyclyl;
R^{2b} is selected from F, Cl, Br, I, NO₂, CN, R^{2b*}, OR^{2b*}, SR^{2b*}, N(R^{2b}+)₂, SO₃R^{2b*}, SO₂R^{2b*}, SO₂N(R^{2b*})₂, C(O)R^{2b*}, C(O)OR^{2b*}, OC(O)R^{2b*}; C(O)N(R^{2b*})₂, N(R^{2b*}) C(O)R^{2b*}, OC(O)N(R^{2b*})₂, N(R^{2b*}) C(O)N(R^{2b*})₂, wherein R^{2b*} is in each case independently selected from hydrogen, C₁₋₈ alkyl, aryl, C₁₋₈heteroaryl, C₃₋₈cycloalkyl, or C₁₋₈heterocyclyl;
provided that at least one of R^{2a} and R^{2b} is other than hydrogen;
R^{z1} is C₁₋₈ alkyl, optionally substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H; and
R^{z3} is C₁₋₈ alkyl, optionally substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H;
provided that the compound of Formula (I) is not any of the following compounds:

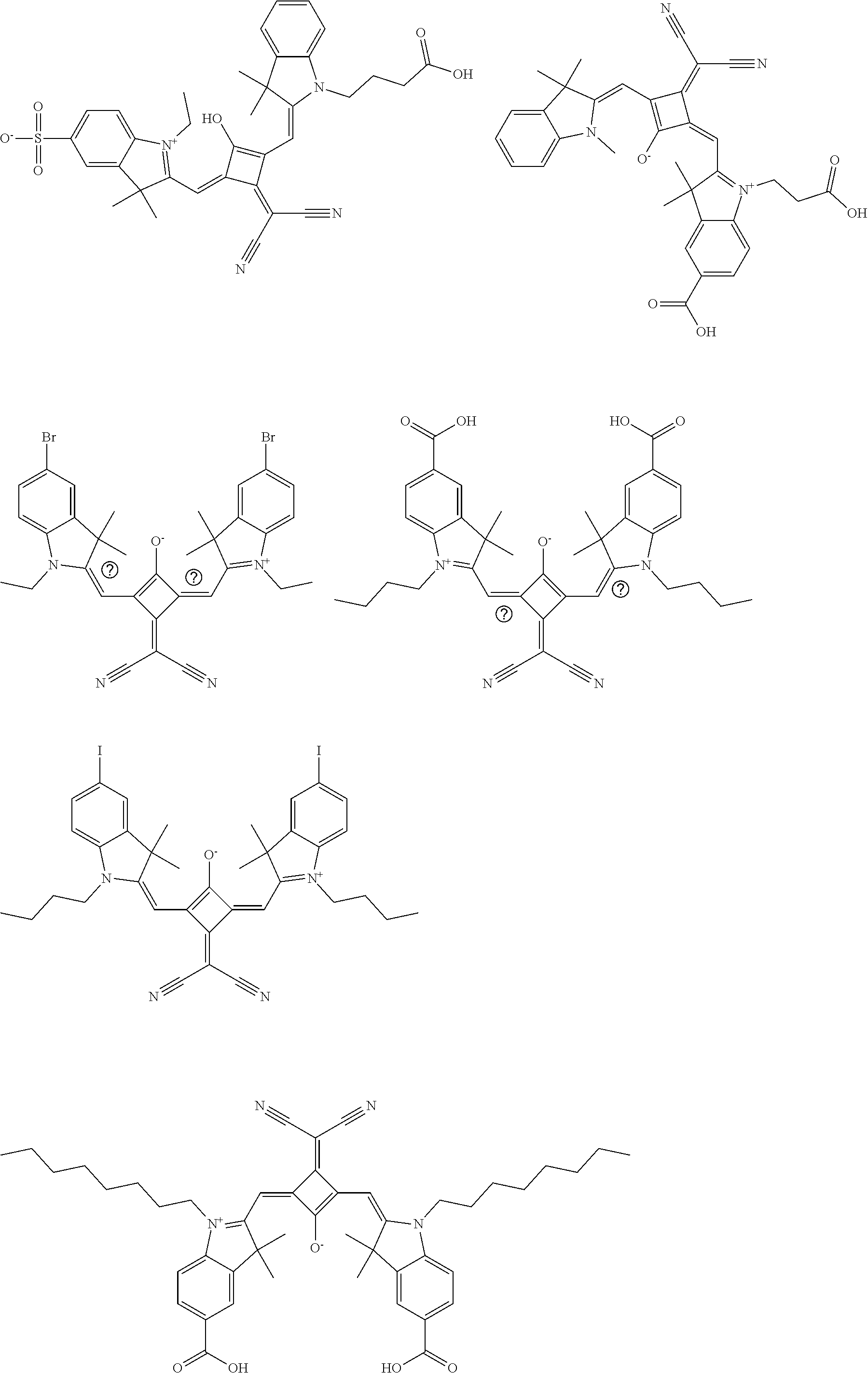

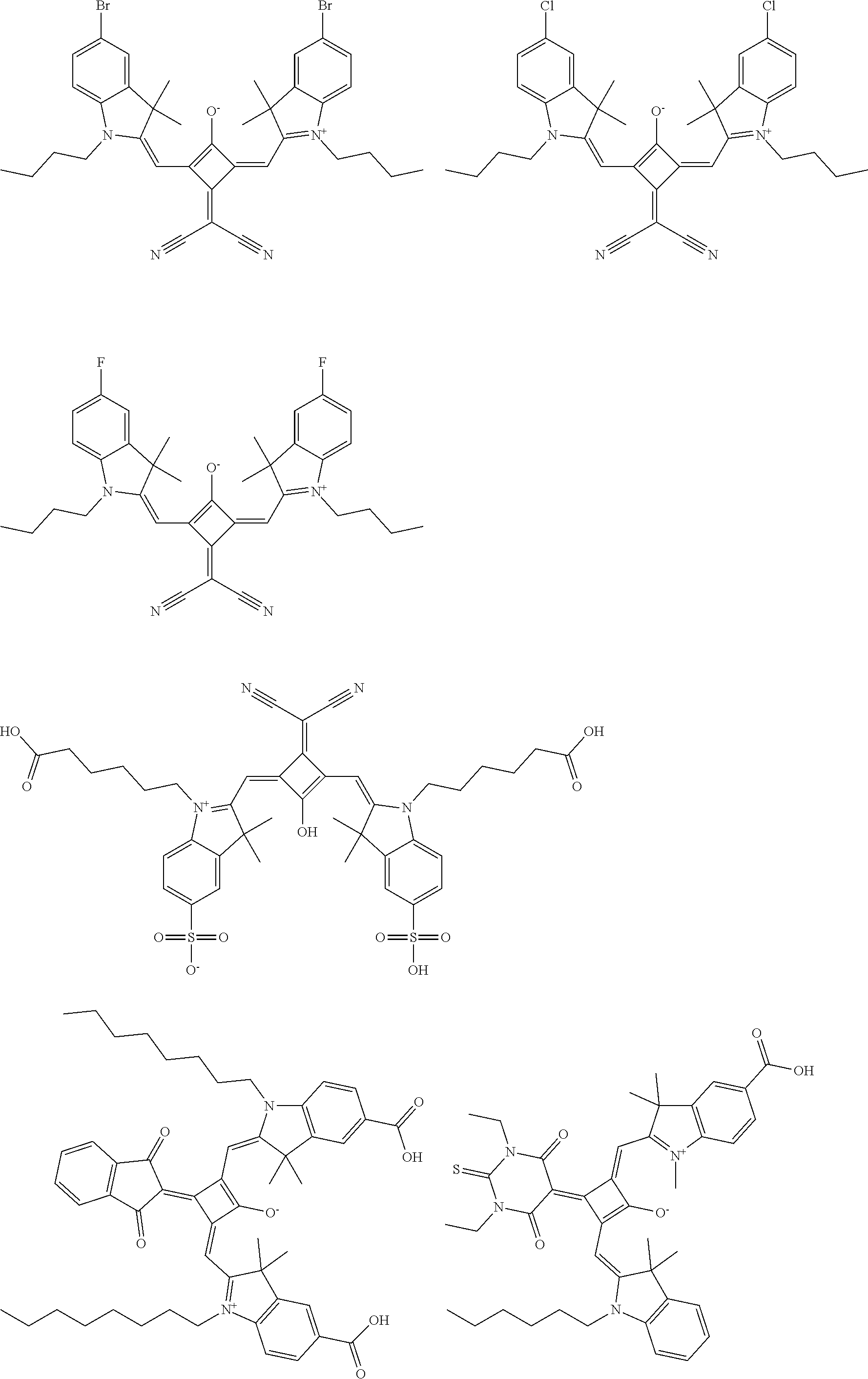

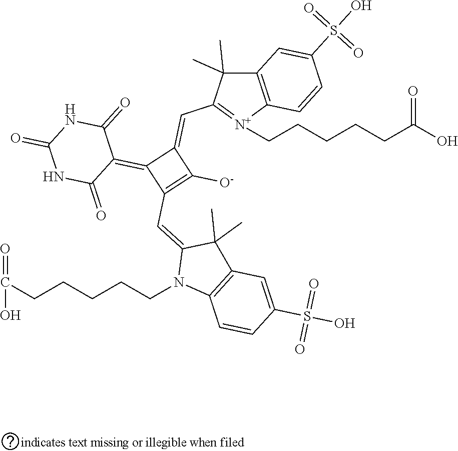 [SMILES: CC1(C)C(/C=C2C([O-])=C(/C=C3/N(CCCCCC(=O)O)c4ccc(S(=O)(=O)O)cc4C3(C)C)C\2=C2C(=O)NC(=O)NC2=O)=[N+](CCCCCC(=O)O)c2ccc(S(=O)(=O)O)cc21]

In some embodiments, Z⁵ is CR^{a}R^{b}.

In some embodiments, R^{a} and R^{b} are each independently selected from CN, F, Cl, CF₃, CHF₂, CHF₂, CO₂H, CONH₂, CO₂C₁₋₃alkyl, COC₁₋₃alkyl, CONHC₁₋₃alkyl, CO₂C₁₋₃haloalkyl, COC₁₋₃haloalkyl, CONHC₁₋₃ haloalkyl, CO₂C₁₋₃alkyl-OH, COC₁₋₃alkyl-OH, and CONHC₁₋₃alkyl-OH.

In some embodiments, R^{a} and R^{b} together form a ring having the formula:

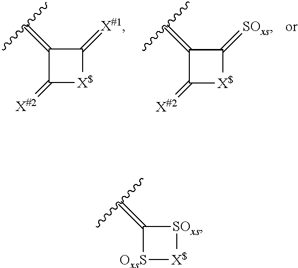

wherein
each xs is independently 1 or 2;
X^{#1} and X^{#2} are each independently selected from oxo, thioxo, imino, and CH₂, provided that both X^{#1} and X^{#2} are not each CH₂;
X^{$} is -ethylene-, -propylene-, -ethenyl-, O-methylene, —NH-methylene-, O-ethylene, —NH-ethylene-, —O-ethylene-O—, —NH-ethylene-O—, —NH-ethylene-NH—, —O-propylene-, —NH-propylene-, —O-propylene-O—, —NH-propylene-O—, —NH-propylene-NH—, aryl, cycloalkyl, heteroaryl, or heterocyclyl, and wherein said methylene, ethylene, ethenyl, propylene, propenyl, aryl, cycloalkyl, heteroaryl, and heterocyclyl may be substituted with 1, 2, or 3 substituents independently selected from F, Cl, Br, I, CN, NH₂, COOH, CONH₂, —(CH₂CH₂O)ₒOCH₃ (o=1-20), —(CH₂CH₂O)ₒH (o=1-20), C₁₋₃ alkyl, C₁₋₃ haloalkyl, C₁₋₃ alkoxy, C₁₋₃ haloalkoxy, C₁₋₃ aminoalkyl, and C₁₋₃ aminohaloalkyl.

In some embodiments, R^{a} and R^{b} together form a ring having the formula:

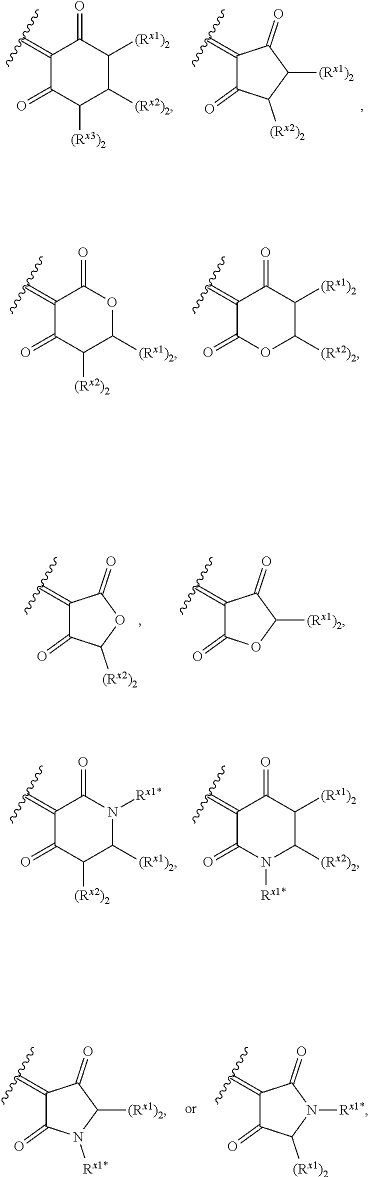

wherein
each R^{x1} is independently selected from H, F, Cl, Br, I, CN, NH₂, COOH, CONH₂, —(CH₂CH₂O)ₒOCH₃ (o=1-20), —(CH₂CH₂O)ₒH (o=1-20), C₁₋₃alkyl, C₁₋₃haloalkyl, C₁₋₃alkoxy, C₁₋₃haloalkoxy, C₁₋₃aminoalkyl, and C₁₋₃aminohaloalkyl; and each R^{x1*} is independently selected from H, CONH₂, —(CH₂CH₂O)ₒOCH₃ (o=1-20), —(CH₂CH₂O).H (o=1-20), C₁₋₃alkyl, and C₁₋₃haloalkyl, or
two geminal R^{x1} groups can together form an oxo, two geminal or adjacent R^{x1} groups can together form a ring, or two adjacent R^{x1} groups can together form a double bond.

In some embodiments, the compound has Formula (I):

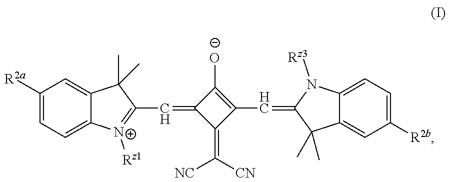

or a pharmaceutically acceptable salt thereof.

In some embodiments, R^{2a} is selected from H, Cl, Br, CN, C₁₋₆ alkoxy, NH₂, NH(C₁₋₆ alkyl), N(C₁₋₈ alkyl)₂, and C(O)OH.

In some embodiments, R^{2a} is selected from Cl, Br, C₁₋₆ alkoxy, and C(O)OH.

In some embodiments, R^{2b} is selected from H, Cl, Br, CN, C₁₋₆ alkoxy, NH₂, NH(C₁₋₆ alkyl), N(C₁₋₈ alkyl)₂, and C(O)OH.

In some embodiments, R^{2b} is selected from Cl, Br, C₁₋₆ alkoxy, and C(O)OH.

In some embodiments:

R^{2a} is selected from Cl, Br, CN, C₁₋₆ alkoxy, NH₂, NH(C₁₋₆ alkyl), N(C₁₋₈ alkyl)₂, and C(O)OH; and

R^{2b} is selected from Cl, Br, CN, C₁₋₆ alkoxy, NH₂, NH(C₁₋₆ alkyl), N(C₁₋₈ alkyl)₂, and C(O)OH.

In some embodiments:
R^{2a} is selected from C₁₆ alkoxy; and
R^{2b} is selected from C₁₋₆ alkoxy and Cl.

In some embodiments:
R^{2a} is hydrogen; and
R^{2b} is selected from Cl, Br, CN, C₁₋₆ alkoxy, NH₂, NH(C₁₋₆ alkyl), N(C₁₋₈ alkyl)₂, and C(O)OH.

In some embodiments, R^{2b} is selected from Cl, Br, and C₁₋₆ alkoxy.

In some embodiments, R^{z1} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H.

In some embodiments, R^{z3} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H.

In some embodiments:
R^{z1} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H;
R^{z3} is C₁₋₈ alkyl, substituted by NH₂, NHC₁₋₃ alkyl, N(C₁₋₃ alkyl)₂, N⁺(C₁₋₃ alkyl)₃, SO₃H, or PO₃H₂.

In some embodiments:
R^{z1} is C₁₋₈ alkyl, substituted by N⁺(C₁₋₃ alkyl)₃; and
R^{z3} is C₁₋₈ alkyl, substituted by N⁺(C₁₋₃ alkyl)₃.

In some embodiments, the compound of Formula (I) is selected from any one of the following compounds:

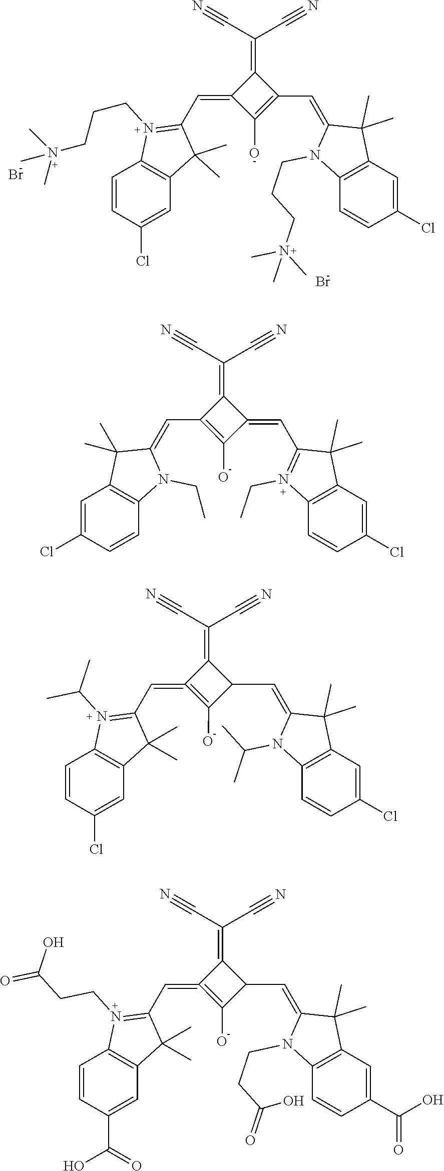

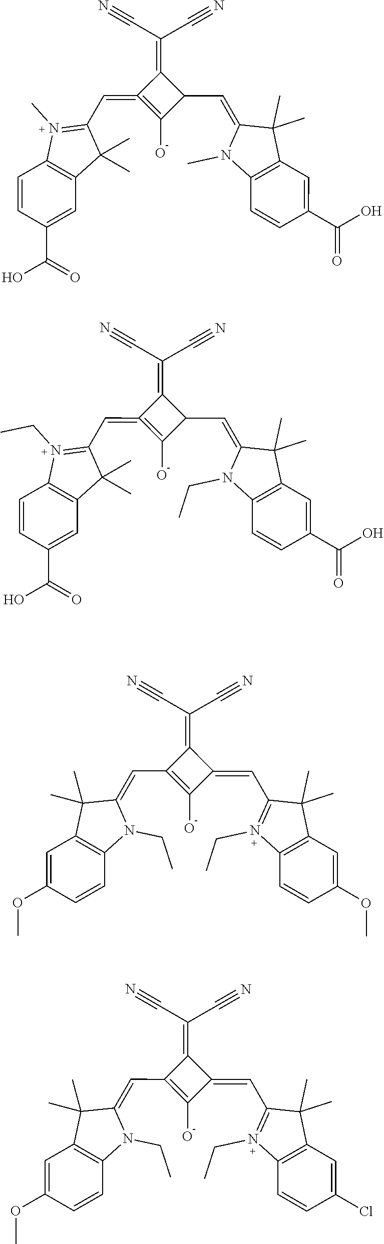

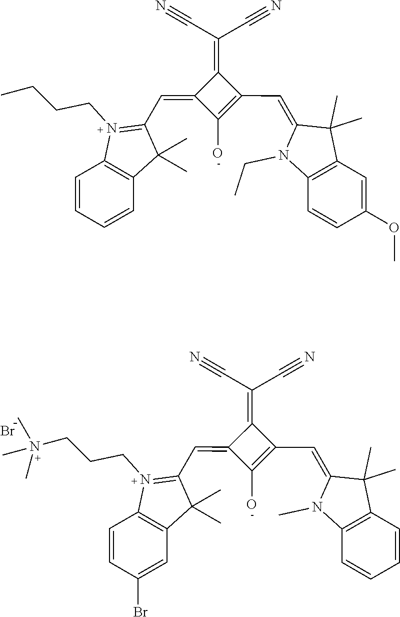

or a pharmaceutically acceptable salt thereof.

In another general aspect, the present disclosure provides a pharmaceutical composition comprising a compound of Formula (II), or a pharmaceutically acceptable salt thereof, and a pharmaceutically acceptable carrier.

In another general aspect, the present disclosure provides a method of imaging a cancerous tumor in a subject, the method comprising:
i) administering to the subject an effective amount of a compound of Formula (II), or a pharmaceutically acceptable salt thereof, or a pharmaceutical composition comprising same;
ii) waiting a time sufficient to allow the compound to accumulate in the cancerous tumor to be imaged; and
iii) imaging the cancerous tumor with a fluorescence imaging technique.

In some embodiments, the method comprises administering the compound orally or intraperitoneally.

In some embodiments, the fluorescence imaging technique is NIR-II fluorescence imaging.

In some embodiments, the time sufficient to allow the compound to accumulate in the cancerous tumor is from about 1 hour to about 168 hours (e.g., about 1 hour, about 2 hours, about 3 hours, or about 4 hours).

In some embodiments, the present disclosure provides a method of treating cancer, the method comprising:
i) imaging a cancerous tumor in a subject according to the imaging method provided herein; and
ii) surgically removing the cancerous tumor from the subject.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 depicts a schematic for the synthesis of symmetrical squaraine fluorophores.

FIG. 2 depicts a schematic for the synthesis of asymmetrical squaraine fluorophores.

FIG. 3 depicts squaraine fluorophores OCTL12-OCTL16.

FIG. 4 depicts a tumor targeting mechanisms for squaraine fluorophores.

FIG. 5 depicts intraoperative imaging of OCTL14 for fluorescence guided surgery (FGS). (a) FGS using the squaraine. Tumor-bearing mice were intravenously injected with 100 nmol of OCTL14. FGS was performed 6 h post-injection. The yellow circle indicates the lesions for which intraoperative image-guided surgeries were performed. The middle panels show the tumor locations before/after surgery and resected tumors of different sizes. Scale bars=1 cm. (b) Clinical impact of the use of OCTL14. The number of tumors detected using color images only was compared to those detected using NIR imaging by experienced surgeons.

FIG. 6 depicts optical properties and stability of squaraine fluorophores.

FIG. 7 depicts (a) Chemical structures of the five asymmetric squaraine derivatives. (b) Quantitative calculation of physicochemical properties of the squaraine fluorophores. (c) Optical properties of the squaraine fluorophores in phosphate-buffered saline (PBS, pH 7.4) with 5% bovine serum albumin (BSA). MW, molecular weight; TPSA, topological polar surface area; HBD, hydrogen bond donors; HBA, hydrogen bond acceptors; Ext. Coeff., extinction coefficient; QY, quantum yield; Abs, absorbance; Fl, fluorescence.

FIG. 8 depicts inhibition assay of cellular uptake of OCTL14 in ID8 cells. Cells were pre-blocked with bromsulphthalein (BSP), cyclosporin A, MK-571, D22, 2DG, oligomycin, Dyngo 4a, Pitstop 2 for 5-30 min or incubated and then incubated with 2 μM of OCTL14 for 30 min. Alternatively, cells were incubated with 2 μM of OCTL14 at 4° C. for 30 min. Scale bar=100 μm.

FIG. 9 depicts (a) Subcellular localization of OCTL14 (10 μM) in ID8 cells was determined after 30 min of incubation at 37° C. Nuclei, mitochondria, or lysosomes were stained with NucBlue™, MitoTracker Green FM, or LysoTracker™ Red DND-99 L7528 and imaged using an epifluorescence microscope. Scale bar=20 μm. (b) Co-localization index of OCTL14 with MitoTracker Green or LysoTracker Red. The index was calculated by dividing the area of overlap between OCTL14 and MitoTracker or LysoTracker by the total area of MitoTracker or LysoTracker, respectively.

### DETAILED DESCRIPTION

The standard treatment for the most common epithelial ovarian cancer is surgery followed by chemotherapy. One of the significant prognostic factors of ovarian cancer patients is the amount of residual tumor after surgery. Therefore, it is crucial to reduce the tumor burden during surgery as much as possible. However, accurate detection, localization, and differentiation of peritoneally-disseminated ovarian cancer from normal tissue are often challenging, which currently relies on visual inspection and palpation. To overcome these limitations, fluorescence-guided surgery (FGS) has emerged as a technique to highlight cancer cells and provide surgeons with real-time image guidance. FGS has been approved for various procedures, including identification of tumor margin, sentinel lymph node mapping, angiography, and lymphography, and improved tumor resection rates and prognosis while minimizing normal tissue damage. In response to this, a number of new imaging methods for FGS have been developed. Amongst these, near-infrared (NIR) imaging features reduced scattering, minimal tissue absorption with high tissue penetration depth, low tissue autofluorescence interference, and is better suited for FGS offering high signal-to-background ratio (SBR) and resolution for imaging of deep tissue.

For FGS of cancer, various tumor-targeting molecules, such as antibodies, nanoparticles, proteins, peptides, and small molecules, have been developed. However, multifunctional nanoparticles showed the unexpected accumulation in vital organs, and their unknown long-term toxicity raises safety concerns. Antibodies targeting tumor biomarkers are generally too large to transfer into tumor tissues and show slow clearance, resulting in a low target-to-background ratio (TBR) and prolonged waiting time after administration. Alternatively, small molecules measuring 10-1000-fold smaller than peptides and proteins, which can rapidly readily diffuse into and accumulate in target tissues and then be rapidly excreted from the system, have been explored to achieve a high TBR. One of such strategies is the use of a ligand for the folate receptor α (FRα), which is overexpressed in ovarian cancer. A recent phase II clinical study evaluated a folate analog conjugated to a NIR fluorescent fluorophore known as OTL38 with promising results. However, a relatively high false-positive rate of 23% was reported because of its off-target binding to FRB. In addition, it still needs a prolonged clearance time after intravenous injection ranging from 2 to 18 hours prior to surgery. Currently, an FDA-approved small molecule NIR probe indocyanine green (ICG) is available in the clinic; however, it shows no specific targeting capability and has significant limitations in FGS of cancer. Contrary, a “structure-inherent targeting (SIT)” strategy has been utilized to improve the biodistribution and targetability of molecular probes by tuning the lipophilicity and charge of backbone structures, allowing for high uptake in specific tissues with low non-specific binding. Hence, the present disclosure provides squaraine chromophores useful, e.g., fast and efficient targeting of ovarian cancers and their FGS. These zwitterionic and planar structured squaraines are water-soluble and stable in aqueous solutions due to the molecule's 3D conformation in which the core is protected, resulting in reduced serum binding and superb optical properties in the NIR wavelengths, allowing surgeons to more precisely perform surgical tumor resection.

### Compounds

The present disclosure provides conjugated squaraine fluorophores and methods of using these fluorophores, e.g., for imaging a cancerous tumor, wherein the conjugated squaraine fluorophores have a general formula:

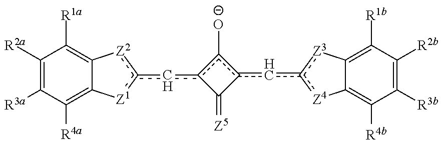

and pharmaceutically acceptable salts thereof, wherein:
Z¹ is N—R^{z1#} and Z² is O, S, C(R^{z2})₂, NR^{z2#}, N—CR^{z2}, or C(R^{z2})—C(R^{z2}); or Z¹ is null and Z² is Z*—N(R^{z2#})—, wherein Z* is N═CR^{z2}, C(R^{z2})—C(R^{z2}); O, S, or C(R^{z2})₂;
Z³ is N—R^{z3#} and Z⁴ is O, S, C(R^{z4})₂, NR^{z4}, N═CR^{z4}, or C(R^{z4})—C(R^{z4}); or Z³ is null and Z⁴ is Z**—N(R^{z4#})—, wherein Z** is N═CR^{z4}, C(R^{z4})═C(R^{z4}), O, S, or C(R^{z4})₂;
R^{z1#}is C₁₋₈alkyl, optionally substituted one or more times by aryl, heteroaryl, COOH, OH, NH₂, NHCH₃, N(CH₃)₂, N(CH₃)₃, PO₃H₂, SO₃H,
R^{z2#}is C₁₋₈alkyl, optionally substituted one or more times by aryl, heteroaryl, COOH, OH, NH₂, NHCH₃, N(CH₃)₂, N(CH₃)₃, PO₃H₂, SO₃H,
R^{z3#}is C₁₋₈alkyl, optionally substituted one or more times by aryl, heteroaryl, COOH, OH, NH₂, NHCH₃, N(CH₃)₂, N(CH₃)₃, PO₃H₂, SO₃H;
R^{z4#}is C₁₋₈alkyl, optionally substituted one or more times by aryl, heteroaryl, COOH, OH, NH₂, NHCH₃, N(CH₃)₂, N(CH₃)₃, PO₃H₂, SO₃H;
R^{z2} is in case independently selected from F, Cl, Br, I, NO₂, CN, R^{z2*}, OR^{z2*} N(R^{z2*})₂, SO₃R^{z2*}, SO₂R^{z2*}, SO₂N(R^{z2*})₂, C(O)R^{z2*}; C(O)OR^{z2*}, OC(O)R^{z2*}; C(O)N(R^{z2*})₂, N(R^{z2*}) C(O)R^{z2*}, OC(O)N(R^{z2*})₂, N(R^{z2*}) C(O)N(R^{z2*})₂, wherein R^{z2}* is in each case independently selected from hydrogen, C₁₋₈alkyl, aryl, C₁₋₈heteroaryl, C₃₋₈cycloalkyl, or C₁₋₈heterocyclyl;
R^{z4} is in case independently selected from F, Cl, Br, I, NO₂, CN, R^{z4*}, OR^{z4*}, N(R^{z4*})₂, SO₃R^{z4*}, SO₂R^{z4*}, SO₂N(R^{z4*})₂, C(O)R^{z4*}; C(O)OR^{z4*}, OC(O)R^{z4*}; C(O)N(R^{z4*})₂, N(R^{z4*}) C(O)R^{z4*}, OC(O)N(R^{z4*})₂, N(R^{z4*}) C(O)N(R^{z4*})₂, wherein R^{z4*} is in each case independently selected from hydrogen, C₁₋₈alkyl, aryl, C₁₋₈heteroaryl, C₃₋₈cycloalkyl, or C₁₋₈heterocyclyl;
R^{1a} is F, Cl, Br, I, NO₂, CN, R^{1a*}, OR^{1a*}, SR^{1a*}, N(R^{1a*})₂, SO₃R^{1a*}, SO₂R^{1a*}, SO₂N(R^{1a*})₂, C(O)R^{1a*}; C(O)OR^{1a*}, OC(O)R^{1a*}; C(O)N(R^{1a*})₂, N(R^{1a*}) C(O)R^{1a*}, OC(O)N(R^{1a*})₂, N(R^{1a*}) C(O)N(R^{1a*})₂, wherein R^{1a*} is in each case independently selected from hydrogen, C₁₋₈alkyl, aryl, C₁₋₈heteroaryl, C₃₋₈cycloalkyl, or C₁₋₈heterocyclyl;
R^{2a} is F, Cl, Br, I, NO₂, CN, R^{2a*}, OR^{2a*}, SR^{2a*}, N(R^{2a*})₂, SO₃R^{2a*}, SO₂R^{2a}* SO₂N(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, N(R^{2a*})C(O)N(R^{z4*})₂, wherein R^{2a*} is in each case independently selected from hydrogen, C₁₋₈alkyl, aryl, C₁₋₈heteroaryl, C₃₋₈cycloalkyl, or C₁₋₈heterocyclyl;
R^{3a} is F, Cl, Br, I, NO₂, CN, R^{3a*}, OR^{3a*}, SR^{3a*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*}) C(O)R^{3a*}, OC(O)N(R^{3a*})₂, N(R^{3a*}) C(O)N(R^{3a*})₂, wherein R^{3a*} is in each case independently selected from hydrogen, C₁₋₈alkyl, aryl, C₁₋₈heteroaryl, C₃₋₈cycloalkyl, or C₁₋₈heterocyclyl;

R^{4a} is F, Cl, Br, I, NO₂, CN, R^{a*}, OR^{4a*}, SR^{4a*}, N(R^{4a*})₂, SO₃R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*}) C(O)R^{4a*}, OC(O)N(R^{4a*})₂, N(R^{4a*}) C(O)N(R^{4a*})₂, wherein R^{4a*} is in each case independently selected from hydrogen, C₁₋₈alkyl, aryl, C₁₋₈heteroaryl, C₃₋₈cycloalkyl, or C₁₋₈heterocyclyl;
wherein any two or more of R^{1a}, R^{2a}, R^{3a}, R^{4a}, R^{z1}, and R^{z2}, may together form a ring;
R^{1b} is F, Cl, Br, I, NO₂, CN, R^{1b*}, OR^{1b*}, SR^{1b*}, N(R^{1b*})₂, SO₃R^{1b*}, SO₂R^{1b*}, SO₂N(R^{1b*})₂, C(O)R^{1b*}; C(O)OR^{1b*}, OC(O)R^{1b*}; C(O)N(R^{1b*})₂, N(R^{1b*}) C(O)R^{1b*} OC(O)N(R^{1b*})₂, N(R^{1b*}) C(O)N(R^{1b*})₂, wherein R^{1b*} is in each case independently selected from hydrogen, C₁₋₈alkyl, aryl, C₁₋₈heteroaryl, C₃₋₈cycloalkyl, or C₁₋₈heterocyclyl;
R^{2b} is F, Cl, Br, I, NO₂, CN, R^{2b*}, OR^{2b*}, SR^{2b*}, N(R^{2b*})₂, SO₃R^{2b*}, SO₂R^{2b*} SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, N(R^{2b*}) C(O)N(R^{2b*})₂, wherein R^{2b*} is in each case independently selected from hydrogen, C₁₋₈alkyl, aryl, C₁₋₈heteroaryl, C₃₋₈cycloalkyl, or C₁₋₈heterocyclyl;
R^{3b} is F, Cl, Br, I, NO₂, CN, R^{3b*}, OR^{3b*}, SR^{3b*}, N(R^{3b*})₂, SO₃R^{3b*}, SO₂R^{3b*}, SO₂N(R^{3b*})₂, C(O)R^{3b*}; C(O)OR^{3b*}, OC(O)R^{3b*}; C(O)N(R^{3b*})₂, N(R^{3b*}) C(O)R^{3b*}, OC(O)N(R^{3b*})₂, N(R^{3b*}) C(O)N(R^{3b*})₂, wherein R^{3b*} is in each case independently selected from hydrogen, C₁₋₈alkyl, aryl, C₁₋₈heteroaryl, C₃₋₈cycloalkyl, or C₁₋₈heterocyclyl;
R^{4b} is F, Cl, Br, I, NO₂, CN, R^{4b*}, OR^{4b*}, SR^{4b*}, N(R^{4b*})₂, SO₃R^{4b*}, SO₂R^{4b*}, SO₂N(R^{4b*})₂, C(O)R^{4b*}; C(O)OR^{4b*}, OC(O)R^{4b*}; C(O)N(R^{4b*})₂, N(R^{4b*}) C(O)R^{4b*}, OC(O)N(R^{4b*})₂, N(R^{4b*}) C(O)N(R^{4b*})₂, wherein R^{4b*} is in each case independently selected from hydrogen, C₁₋₈alkyl, aryl, C₁₋₈heteroaryl, C₃₋₈cycloalkyl, or C₁₋₈heterocyclyl;
wherein any two or more of R^{1b}, R^{2b}, R^{3b}, R^{4b}, R^{z3}, and R^{z4}, may together form a ring;
Z⁵ is O, S, NR^{a}, or CR^{a}R^{b}, wherein
R^{a} is F, Cl, Br, I, NO₂, CN, R^{a1}, OR^{a1}, SR^{a1}, N(R^{a1})₂, SO₂R^{a1}, SO₂N(R^{a1})₂, C(O)R^{a1}; C(O)OR^{a1}, OC(O)R^{a1}; C(O)N(R^{a1})₂, N(R^{a1})C(O)R^{a1}, OC(O)N(R^{a1})₂, N(R^{a1})C(O)N(R^{a1})₂, wherein R^{a1} is in each case independently selected from hydrogen, C₁₋₈alkyl, aryl, C₁₋₈heteroaryl, C₃₋₈cycloalkyl, or C₁₋₈heterocyclyl; and
R^{b} is F, Cl, Br, I, NO₂, CN, R^{b1}, OR^{b1}, SR^{b1}, N(R^{b1})₂, SO₂R^{b1}, SO₂N(R^{b1})₂, C(O)R^{b1}; C(O)OR^{b1}, OC(O)R^{b1}; C(O)N(R^{b1})₂, N(R^{b1}) C(O)R^{b1}, OC(O)N(R^{b1})₂, N(R^{b1}) C(O)N(R^{b1})₂, wherein R^{b1} is in each case independently selected from hydrogen, C₁₋₈alkyl, aryl, C₁₋₈heteroaryl, C₃₋₈cycloalkyl, or C₁₋₈heterocyclyl;
wherein R^{a} and R^{b} may together form a ring.

In some embodiments, any of the squaraine compounds of this disclosure may exist in one or more different configurations:

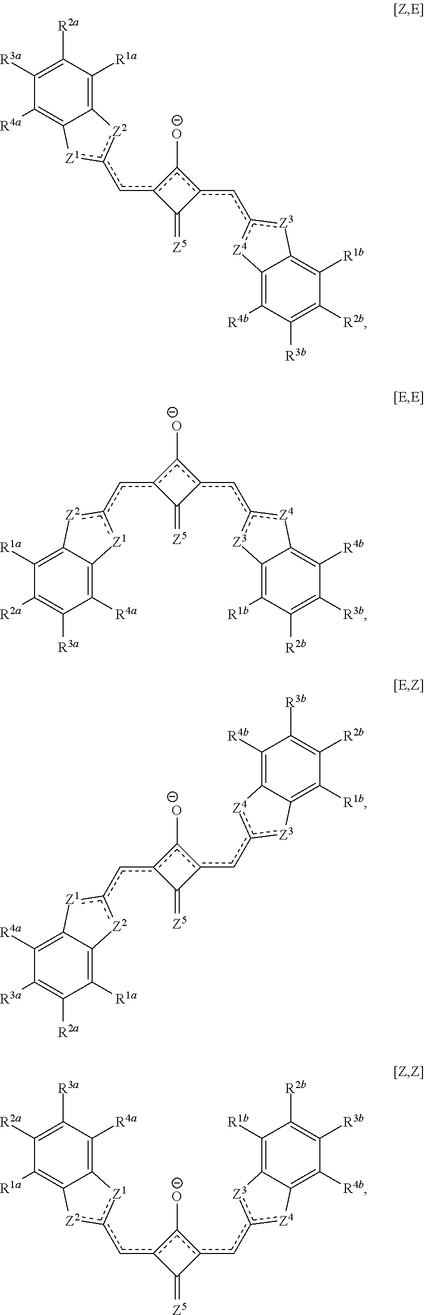

Unless specified to the contrary, the depiction of the squaraine compound with a specific configuration is intended to cover all possible geometric configurations.

However, such as depiction is not intended to convey a measure of purity, such a depiction equally covers a mixture of different isomers as well as a single isomer.

In certain embodiments, the squaraine is predominantly in the [E,E] configuration, i.e., at least 75%, at least 80%, at least 85%, at least 90%, at least 95%, at least 97.5%, or at least 99% on a mol % of the total squaraine content, is in the [E,E] configuration.

In certain embodiments, the squaraine is predominantly in the [E,Z] configuration, i.e., at least 75%, at least 80%, at least 85%, at least 90%, at least 95%, at least 97.5%, or at least 99% on a mol % of the total squaraine content, is in the [E,Z] configuration.

In certain embodiments, the squaraine is predominantly in the Z,E] configuration, i.e., at least 75%, at least 80%, at least 85%, at least 90%, at least 95%, at least 97.5%, or at least 99% on a mol % of the total squaraine content, is in the [Z,E] configuration.

In certain embodiments, the squaraine is predominantly in the [Z,Z] configuration, i.e., at least 75%, at least 80%, at least 85%, at least 90%, at least 95%, at least 97.5%, or at least 99% on a mol % of the total squaraine content, is in the [Z,Z] configuration.

Without being bound by any theory, the skilled person understands that the compounds disclosed herein may be depicted in one of several resonance forms, e.g.:

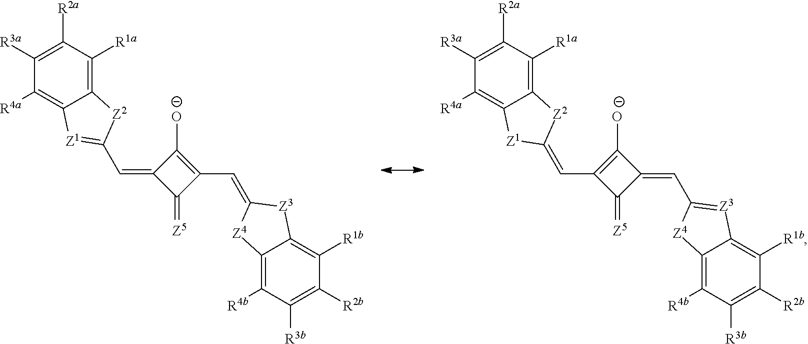

The depiction of one resonance form is intended to cover all possible other resonance forms as well.

In certain embodiments, the squaraine compound is zwitterionically balanced. As used herein, “zwitterionically balanced” means that there are an equal number of cationically and anionically charged functional groups covalently bound within the molecule.

For example, the compound:

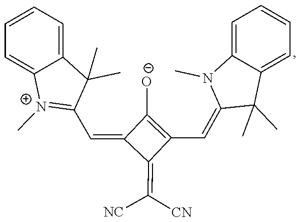

is zwitterionically balanced because it is electrically neutral without requiring any additional pharmaceutically acceptable anionic species. By way of comparison, the compound:

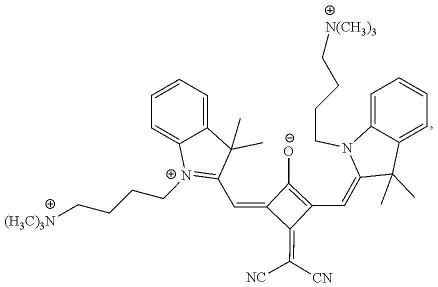

is not zwitterionically balanced (three cationic groups and one anionic group), and requires one or more pharmaceutically acceptable anionic species in order to be electrically neutral. Exemplary anionic species include chloride, bromide, iodide, sulfate, acetate, phosphate, citrate, etc.

In preferred embodiments, Z⁵ is CR^{a}R^{b}. In further embodiments, R^{a} and R^{b} are the same and are selected from CN, F, Cl, CF₃, CHF₂, CHF₂, CO₂H, CONH₂, CO₂C₁₋₃alkyl, COC₁₋₃alkyl, CONHC₁₋₃alkyl, CO₂C₁₋₃haloalkyl, COC₁₋₃haloalkyl, CONHC₁₋₃ haloalkyl, CO₂C₁₋₃alkyl-OH, COC₁₋₃alkyl-OH, CONHC₁₋₃alkyl-OH.

In certain embodiments, R^{a} and R^{b} can together form a ring having the formula:

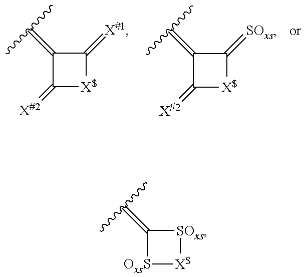

wherein xs is independently selected from 1 (sulfoxide) or 2 (sulfone);
X^{#1} and X^{#2} are independently selected from oxo, thioxo, imino, and CH₂, provided that both X^{#1} and X^{#2} are not each CH₂;
X^{$} is -ethylene-, -propylene-, -ethenyl-, O-methylene, —NH-methylene-, O-ethylene, —NH-ethylene-, —O-ethylene-O—, —NH-ethylene-O—, —NH-ethylene-NH—, —O-propylene-, —NH-propylene-, —O-propylene-O—, —NH-propylene-O—, —NH-propylene-NH—, aryl, cycloalkyl, heteroaryl, or heterocyclyl, wherein said methylene, ethylene, ethenyl, propylene, propenyl, aryl, cycloalkyl, heteroaryl, and heterocyclyl may be substituted one or more times by F, Cl, Br, I, CN, NH₂. COOH, CONH₂, —(CH₂CH₂O)ₒOCH₃ (o=1-20), —(CH₂CH₂O)ₒH (o=1-20), C₁₋₃alkyl, C₁₋₃haloalkyl, C₁₋₃alkoxy, C₁₋₃haloalkoxy, C₁₋₃aminoalkyl, or C₁₋₃aminohaloalkyl.

When R^{a} and R^{b} together form a ring, exemplary ring systems include:

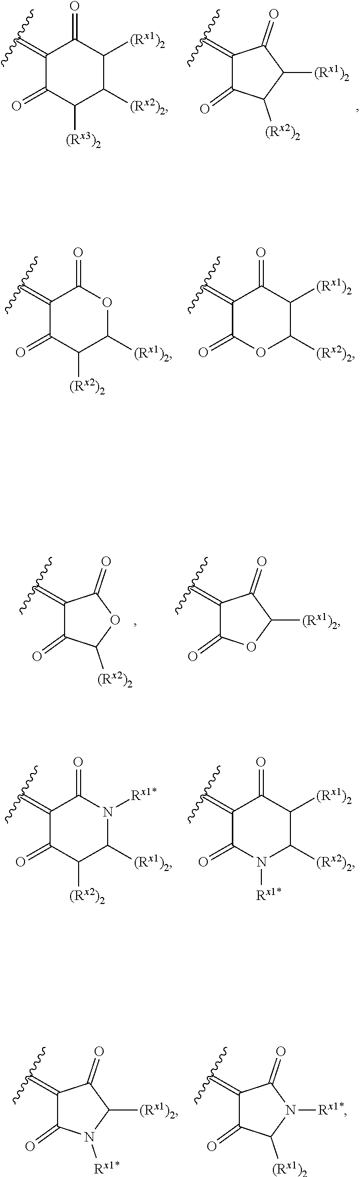

wherein
R^{x1} is in each case independently selected from H, F, Cl, Br, I, CN, NH₂, COOH, CONH₂, —(CH₂CH₂O)ₒCH₃ (o=1-20), —(CH₂CH₂O)ₒH (o=1-20), C₁₋₃alkyl, C₁₋₃haloalkyl, C₁₋₃alkoxy, C₁₋₃haloalkoxy, C₁₋₃aminoalkyl, or C₁₋₃aminohaloalkyl.

R^{x1*} is in each case independently selected from H, CONH₂, —(CH₂CH₂O)ₒOCH₃ (o=1-20), —(CH₂CH₂O)ₒH (o=1-20), C₁₋₃alkyl, or C₁₋₃haloalkyl,
wherein two geminal R^{x1} groups can together form an oxo, wherein two geminal or adjacent R^{x1} groups can together form a ring, and wherein two adjacent R^{x1} groups can together form a double bond.

When R^{a} and R^{b} together form a ring, said ring can be symmetrical or asymmetrical. A symmetrical ring system has an internal line of symmetry along the bond leading to the cyclobutene:

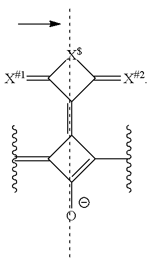

The squaraine compound can asymmetrical, semi-symmetrical, or symmetrical. As used herein, an asymmetrical squaraine C₁₋₃alkyl is a compound in which the two heterocyclic bases are not the same.

In some embodiments, the squaraine fluorophore can be an asymmetrical squaraine wherein each of R^{1a}, R^{2a}, R^{3a}, and R^{4a} are hydrogen, and at least one of R^{1b} R^{2b}, R^{3b}, or R^{4b} are not hydrogen, i.e., a non-hydrogen substituent. In such embodiments, the non-hydrogen substituent can be selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹- is selected from null, CH₂, or CH₂CH₂.

In certain embodiments, each of R^{1a}, R^{2a}, R^{3a}, and R^{4a} are hydrogen, R^{2b} is selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, and each of R^{2a}, R^{2c}, and R^{2d} are hydrogen.

In certain embodiments, each of R^{1a}, R^{2a}, R^{3a}, and R^{4a} are hydrogen, R^{4b} is selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, and each of R^{2a}, R^{2b}, and R^{2c} are hydrogen.

In certain embodiments, each of R^{1a}, R^{2a}, R^{3a}, and R^{4a} are hydrogen, R^{2b} and R^{4b} are independently selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, and each of R^{1b} and R^{3b} are hydrogen.

In certain embodiments, each of R^{1a}, R^{2a}, R^{3a}, and R^{4a} are hydrogen, R^{2b} is selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, and each of R^{1b}, R^{3b}, and R^{4b} are hydrogen.

In certain embodiments, Z¹ is NC₁₋₆alkyl, each of R^{1a}, R^{2a}, R^{3a}, and R^{4a} are hydrogen, R^{3b} is selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N(C₁₋₃alkyl)₃, Q¹-SO₃H, Q¹-PO₃H₂, and Q¹-CO₂H, and each of R^{1b}, R^{2b}, and R^{4b} are hydrogen.

In some embodiments, the asymmetrical squaraine will have the same Z¹ and Z³ substituents. In such embodiments, Z¹ and Z³ can each be NC₁₋₆alkyl, optionally substituted one or more times by F, Cl, Br, I, CN, C₁₋₃alkyl, C₁₋₃haloalkyl, OH, OC₁₋₃alkyl, NH₂, NHC₁₋₃alkyl, N(C₁₋₃alkyl)₂, N″ (C₁₋₃alkyl)₃, SO₃H, PO₃H₂, and CO₂H. In other embodiments, Z¹ and Z³ are not the same. For example, one of Z¹ and Z³ is NC₁₋₆alkyl, wherein the alkyl group is not substituted, and the other of Z¹ and Z³ is NC₁₋₆alkyl, wherein the alkyl group is substituted, for example substituted one or more times by F, Cl, Br, I, CN, C₁₋₃alkyl, C₁₋₃haloalkyl, OH, OC₁₋₃alkyl, NH₂, NHC₁₋₃alkyl, N(C₁₋₃alkyl)₂, N″ (C₁₋₃alkyl)₃, SO₃H, PO₃H₂, and CO₂H, preferably N⁺(C₁₋₃alkyl)₃. In some instances, the asymmetrical squaraine is a compound in which each of R^{1a}, R^{2a}, R^{3a}, and R^{4a} are hydrogen, least one of R^{1b}, R^{2b}, R^{3b}, or R^{4b} are not hydrogen, and Z¹ is NC₁₋₆alkyl, wherein the alkyl group is not substituted. In other instances, the asymmetrical squaraine is a compound in which each of R^{1a}, R^{2a}, R^{3a} and R^{4a} are hydrogen, least one of R^{1b}, R^{2b}, R^{3b}, or R^{4b} are not hydrogen, and Z¹ is NC₁₋₆alkyl, wherein the alkyl group is substituted, for example substituted one or more times by F, Cl, Br, I, CN, C₁₋₃alkyl, C₁₋₃haloalkyl, OH, OC₁₋₃alkyl, NH₂, NHC₁₋₃alkyl, N(C₁₋₃alkyl)₂, N″ (C₁₋₃alkyl)₃, SO₃H, PO₃H₂, and CO₂H, preferably N(C₁₋₃alkyl)₃.

In other embodiments, Z¹ and Z³ can each be NC₁₋₆alkyl, wherein in both cases C₁₋₆alkyl is substituted one or more times by F, Cl, Br, I, CN, C₁₋₃alkyl, C₁₋₃haloalkyl, OH, OC₁₋₃alkyl, NH₂, NHC₁₋₃alkyl, N(C₁₋₃alkyl)₂, N⁺(C₁₋₃alkyl)₃, SO₃H, PO₃H₂, and CO₂H, preferably N⁺(C₁₋₃alkyl)₃. In certain embodiments, Z¹ and Z³ are both NC₁₋₆alkyl, substituted one or more times by N⁺(C₁₋₃alkyl)₃, SO₃H, PO₃H₂, or CO₂H. In some embodiments, one of Z¹ and Z³ is NC₁₋₆alkyl, substituted one or more times by SO₃H, PO₃H₂, or CO₂H, and the other of Z¹ and Z³ is NC₁₋₆alkyl, one or more times by N″ (C₁₋₃alkyl)₃. In some such embodiments, one of Z¹ and Z³ is NC₁₋₆alkyl, substituted once by CO₂H, and the other one of Z¹ and Z³ is NC₁₋₆alkyl, substituted once by N⁺(C₁₋₃alkyl)₃.

A semi-symmetrical squaraine is a compound in which the heterocyclic bases are the same (i.e., Z¹=Z³, Z²=Z⁶, R^{1a}=R^{1b}, R^{2a}=R^{2b}, R^{3a}=R^{3b}, and R^{4a}=R^{4b}, but R^{a} and R^{b} are not the same. A symmetrical squaraine is a compound in which the heterocyclic bases as well as R^{a} and R^{b} are the same (including compounds in which R^{a} and R^{b} together form a symmetrical ring system).

For symmetrical and semi-symmetrical squaraine fluorophores, certain definitions may be collapsed, for example R^{1a}=R^{1b}=R¹, R^{2a}=R^{2b}=R², R^{3a}=R^{3b}=R³, and R^{4a}=R^{4b}=R⁴.

Symmetrical and semi-symmetrical squaraine fluorophores include examples one of R¹, R², R³ and R⁴ is an electron-withdrawing group, and the remaining three of R¹, R², R³ and R⁴ are each hydrogen. Exemplary electron-withdrawing groups include F, Cl, Br, I, NO₂, CN, SO₂R^{1a}, SO₂N(R^{1a})₂, C(O)R^{1a}, C(O)OR^{1a}, and C(O)N(R^{1a})₂ (here R^{1a} groups, defined above, are used in the exemplary, not limiting, fashion). In some embodiments the electron-withdrawing group is an alkyl group substituted with one or more of F, Cl, Br, I, NO₂, CN, SO₂R^{1a}, SO₂N(R^{1a})₂, C(O)R^{1a}, C(O)OR^{1a}, and C(O)N(R^{1a})₂. Preferable electron-withdrawing groups include F, Cl, Br, I, C₁₋₃haloalkyl, C(O) C₁₋₃alkyl, C(O)OC₁₋₃alkyl, C(O)NHC₁₋₃alkyl, C(O)OH, SO₃H, C(O) C₁₋₃haloalkyl, C(O)OC₁₋₃haloalkyl, and C(O)NHC₁₋₃haloalkyl.

In some embodiments, R¹ is an electron-withdrawing group as defined herein and each of R², R³, and R⁴ are hydrogen.

In some embodiments, R² is an electron-withdrawing group as defined herein and each of R¹, R³, and R⁴ are hydrogen.

In some embodiments, R³ is an electron-withdrawing group as defined herein and each of R¹, R², and R⁴ are hydrogen.

In some embodiments, R⁴ is an electron-withdrawing group as defined herein and each of R¹, R², and R³ are hydrogen.

In some embodiments, R¹ and R³ are independently electron-withdrawing group as defined herein and each of R², and R⁴ are hydrogen.

In some embodiments, R² and R⁴ are independently electron-withdrawing group as defined herein and each of R¹, and R³ are hydrogen.

In certain embodiments, when Z² and Z⁴ are N—R^{zd}, Z¹ and Z³ are O, S, or C(CH₃)₂, R³ is an electron-withdrawing group as defined herein and each of R¹, R², and R⁴ are hydrogen. In certain embodiments, when Z² and Z⁴ are N—R_{ad}, Z¹ and Z³ are O, S, or C (CH₃)₂, R¹ is an electron-withdrawing group as defined herein and each of R², R³, and R⁴ are hydrogen. In other embodiments, R¹ and R³ are electron-withdrawing groups as defined herein and R² and R⁴ are hydrogen. In certain embodiments, when Z² and Z⁴ are N—R^{zd}, Z¹ and Z³ are O, S, or C (CH₃)₂, R¹ and R³ are electron-withdrawing groups as defined herein and R² and R⁴ are hydrogen.

In certain embodiments, when Z² and Z⁴ are null, R² is an electron-withdrawing group as defined herein and each of R², R³, and R⁴ are hydrogen. In certain embodiments, when Z² and Z⁴ are null, R⁴ is an electron-withdrawing group as defined herein and each of R¹, R², and R³ are hydrogen. In other embodiments, R² and R⁴ are electron-withdrawing groups as defined herein and R¹ and R³ are hydrogen. In certain embodiments, when Z² and Z⁴ are null, R² and R⁴ are electron-withdrawing groups as defined herein and R¹ and R³ are hydrogen.

In other embodiment, one of R¹, R², R³ and R⁴ is an electron-donating group, and the remaining three of R¹, R², R³ and R⁴ are each hydrogen. Exemplary electron-donating groups include OR^{1a} and N(R^{1a})₂ (here R^{1a} groups, defined above, are used in the exemplary, not limiting, fashion). Preferable electron-donating groups include OH, NH₂, OC₁₋₃alkyl, NHC₁₋₃alkyl, N(C₁₋₃alkyl)₂, OC₁₋₃alkyl, NHC₁₋₃alkyl, and N(C₁₋₃alkyl)₂, as well as unsubstituted alkyl and aryl groups, and alkyl and aryl groups substituted with one or more of OH, NH₂, OC₁₋₃alkyl, NHC₁₋₃alkyl, N(C₁₋₃alkyl)₂, OC₁₋₃alkyl, NHC₁₋₃alkyl, and N(C₁₋₃alkyl)₂.

In some embodiments, R¹ is an electron-donating group as defined herein and each of R², R³, and R⁴ are hydrogen.

In some embodiments, R² is an electron-donating group as defined herein and each of R¹, R³, and R⁴ are hydrogen.

In some embodiments, R³ is an electron-donating group as defined herein and each of R¹, R², and R⁴ are hydrogen.

In some embodiments, R⁴ is an electron-donating group as defined herein and each of R¹, R², and R³ are hydrogen.

In some embodiments, R¹ and R³ are independently electron-donating group as defined herein and each of R², and R⁴ are hydrogen.

In some embodiments, R² and R⁴ are independently electron-donating group as defined herein and each of R¹, and R³ are hydrogen.

In certain embodiments, when Z² and Z⁴ are N—R^{zd}, Z¹ and Z³ are O, S, or C (CH₃)₂, R³ is an electron-donating group as defined herein and each of R¹, R², and R⁴ are hydrogen. In certain embodiments, when Z² and Z⁴ are N—R_{zd}, Z¹ and Z³ are O, S, or C (CH₃)₂, R¹ is an electron-donating group as defined herein and each of R², R³, and R⁴ are hydrogen. In other embodiments, R¹ and R³ are electron-donating groups as defined herein and R² and R⁴ are hydrogen. In certain embodiments, when Z² and Z⁴ are N—R^{zd}, Z¹ and Z³ are O, S, or C (CH₃)₂, R¹ and R³ are electron-donating groups as defined herein and R² and R⁴ are hydrogen.

In certain embodiments, when Z² and Z⁴ are null, R² is an electron-donating group as defined herein and each of R², R³, and R⁴ are hydrogen. In certain embodiments, when Z² and Z⁴ are null, R⁴ is an electron-donating group as defined herein and each of R¹, R², and R³ are hydrogen. In other embodiments, R² and R⁴ are electron-donating groups as defined herein and R¹ and R³ are hydrogen. In certain embodiments, when Z² and Z⁴ are null, R² and R⁴ are electron-donating groups as defined herein and R¹ and R³ are hydrogen.

In some embodiments, R^{zd} is C₁₋₈alkyl, optionally substituted one or more times with aryl, OH, NH₂, NHC₁₋₃alkyl, N(C₁₋₃alkyl)₂, N⁺(C₁₋₃alkyl)₃, X⁻, SO₃H, CO₂H, wherein X⁻ is a pharmaceutically acceptable anion. Exemplary R^{zd} groups include methyl, ethyl, benzyl, propyl, n-butyl, n-pentyl, n-hexyl, n-heptyl, n-octyl, 2-hydroxyethyl, 3-hydroxypropyl, 4-hydroxybutyl, 2-aminoethyl, 3-aminopropyl, 4-aminobutyl, 2-phenylethyl, 3-phenylpropyl, 4-phenylbutyl, 2-(dimethylamino)ethyl, 3-(dimethylamino) propyl, 4-(dimethylamino) butyl, 2-(trimethylammonium iodide) ethyl, 3-(trimethylammonium iodide) propyl, 4-(trimethylammonium iodide) butyl, 2-ethanesulfonic acid, 3-propanesulfonic acid, and 4-butanesulfonic acid. The iodide anion may be replaced with another acceptable anion, including bromide, chloride, sulfate, citrate, acetate, etc.

In some embodiments, the squaraine fluorophore compound of this disclosure has Formula (II):

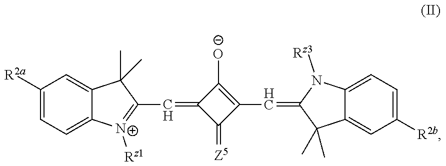

or a pharmaceutically acceptable salt thereof.

In some embodiments, the squaraine fluorophore compound of this disclosure has Formula (I):

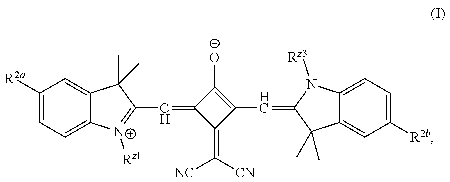

or a pharmaceutically acceptable salt thereof.

In some embodiments:
Z⁵ is O, S, NR^{a}, or CR^{a}R^{b},
R^{a} is F, Cl, Br, I, NO₂, CN, R^{a1}, OR^{a1}, SR^{a1}, N(R^{a1})₂, SO₂R^{a1}, SO₂N(R^{a1})₂, C(O)R^{a1}, C(O)OR^{a1}, OC(O)R^{a1}, C(O)N(R^{a1})₂, N(R^{a1})C(O)R^{a1}, OC(O)N(R^{a1})₂, or N(R^{a1})C(O)N(R^{a1})₂, wherein each R^{a1} is independently selected from hydrogen, C₁₋₈ alkyl, aryl, C₁₋₈ heteroaryl, C₃₋₈ cycloalkyl, and C₁₋₈ heterocyclyl; and
R^{b} is F, Cl, Br, I, NO₂, CN, R^{b1}, OR^{b1}, SR^{b1}, N(R^{b1})₂, SO₂R^{b1}, SO₂N(R^{b1})₂, C(O)R^{b1}, C(O)OR^{b1}, OC(O)R^{b1}, C(O)N(R^{b1})₂, N(R^{b1}) C(O)R^{b1}, OC(O)N(R^{b1})₂, or N(R^{b1}) C(O)N(R^{b1})₂, wherein each R^{b1} is independently selected from hydrogen, C₁₋₈ alkyl, aryl, C₁₋₈ heteroaryl, C₃₋₈ cycloalkyl, and C₁₋₈ heterocyclyl;
or R^{a} and R^{b} together form a ring;
R^{2a} is selected from F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, and N(R^{2a*})C(O)N(R^{2a*})₂, wherein R^{2a*} is in each case independently selected from hydrogen, C₁₋₈ alkyl, aryl, C₁₋₈ heteroaryl, C₃₋₈ cycloalkyl, and C₁₋₈ heterocyclyl;
R^{2b} is selected from F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, N(R^{2b*}) C(O)N(R^{2b*})₂, wherein R^{2b*} is in each case independently selected from hydrogen, C₁₋₈ alkyl, aryl, C₁₋₈heteroaryl, C₃₋₈cycloalkyl, or C₁₋₈heterocyclyl;
R^{z1} is C₁₋₈ alkyl, optionally substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H; and
R^{z3} is C₁₋₈ alkyl, optionally substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H.

In some embodiments, at least one of R^{2a} and R^{2b} is other than hydrogen. In some embodiments, R^{2a} is other than hydrogen. In some embodiments, R^{2b} is other than hydrogen.

In some embodiments, the compound of Formula (II) is not any one of the following compounds:

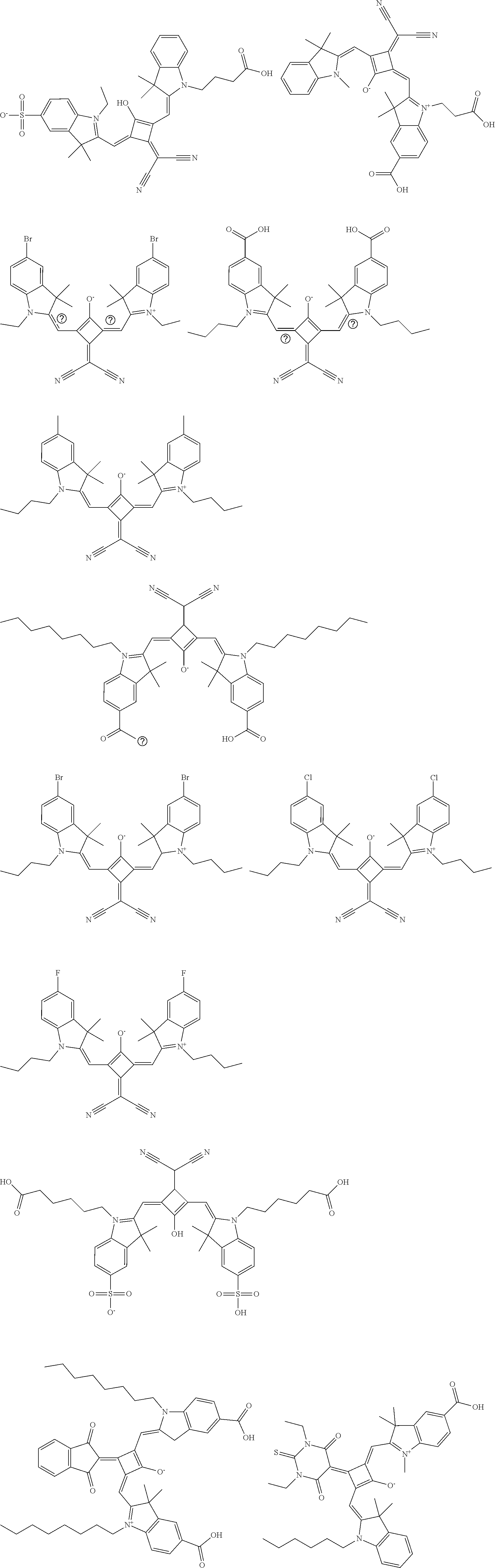

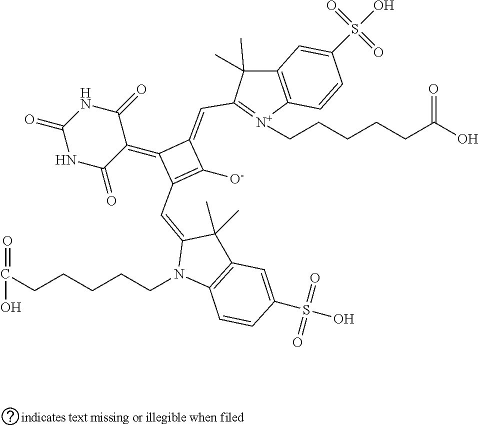 [SMILES: CC1(C)C(/C=C2C([O-])=C(/C=C3/N(CCCCCC(=O)O)c4ccc(S(=O)(=O)O)cc4C3(C)C)C\2=C2C(=O)NC(=O)NC2=O)=[N+](CCCCCC(=O)O)c2ccc(S(=O)(=O)O)cc21]

In some embodiments, R^{2a} is selected from F, Cl, Br, I, NO₂, CN, R^{2a*}, OR^{2a*}, SR^{2a*}, N(R^{2a*})₂, SO₃R^{2a*}, SO₂R^{2a*}, SON(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, and N(R^{2a*})C(O)N(R^{2a*})₂, wherein R^{2a*} is in each case independently selected from hydrogen, C₁₋₈ alkyl, aryl, C₁₋₅ heteroaryl, C₃₋₈ cycloalkyl, and C₁₋₈ heterocyclyl.

In some embodiments, R^{2a} is selected from H, Cl, Br, CN, C₁₋₆ alkoxy, NH₂, NH(C₁₋₆ alkyl), N(C₁₋₈ alkyl)₂, and C(O)OH. In some embodiments, R^{2a} is H. In some embodiments, R^{2a} is selected from F, I, Cl, and Br. In some embodiments, R^{2a} is F. In some embodiments, R^{2a} is C₁. In some embodiments, R^{2a} is I. In some embodiments, R^{2a} is Br. In some embodiments, R^{2a} is selected from OR^{2a*}, SR^{2a*}, N(R^{z2*})₂, SO₃R^{2a*}, SO₂R^{2a*}, SO₂N(R^{2a*})₂, C(O)R^{2a*}; C(O)OR^{2a*}, OC(O)R^{2a*}, C(O)N(R^{2a*})₂, N(R^{2a*})C(O)R^{2a*}, OC(O)N(R^{z2*})₂, and N(R^{2a*})C(O)N(R^{2a*})₂. In some embodiments, R^{2a} is C₁₋₆ alkoxy. In some embodiments, R^{2a} is C(O)OH.

In some embodiments, R^{2a} is selected from F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, and N(R^{2a*})C(O)N(R^{z2*})₂, wherein R^{2a*} is in each case independently selected from hydrogen, C₁₋₈ alkyl, aryl, C₁₋₈ heteroaryl, C₃₋₈ cycloalkyl, and C₁₋₈ heterocyclyl. In some embodiments, R^{2a} is selected from Cl, Br, C₁₋₆ alkoxy, and C(O)OH.

In some embodiments, R^{2b} is selected from F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, and N(R^{2b*}) C(O)N(R^{2b*})₂, wherein R^{2b*} is in each case independently selected from hydrogen, C₁₋₈ alkyl, aryl, C₁₋₈ heteroaryl, C₃₋₈ cycloalkyl, and C₁₋₈ heterocyclyl.

In some embodiments, R^{2b} is selected from H, Cl, Br, CN, C₁₋₆ alkoxy, NH₂, NH(C₁₋₆ alkyl), N(C₁₋₈ alkyl)₂, and C(O)OH. In some embodiments, R^{2b} is H. In some embodiments, R^{2b} is selected from F, I, Cl, and Br. In some embodiments, R^{2b} is F. In some embodiments, R^{2b} is C₁. In some embodiments, R^{2b} is I. In some embodiments, R^{2b} is Br. In some embodiments, R^{2b} is selected from OR^{2b*}, SR^{2b*}, N(R^{2b*})₂, SO₃R^{2b*}, SO₂R^{2b*}, SO₂N(R^{2b*})₂, C(O)R^{2b*}, C(O)OR^{2b*}, OC(O)R^{2b*}, C(O)N(R^{2b*})₂, N(R^{2b*}) C(O)R^{2b*}, OC(O)N(R^{2b*})₂, and N(R^{2b*}) C(O)N(R^{2b*})₂. In some embodiments, R^{2b} is C₁₋₆ alkoxy. In some embodiments, R^{2b} is C(O)OH.

In some embodiments, R^{2b} is selected from F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, and N(R^{2b*}) C(O)N(R^{2b*})₂, wherein R^{2b*} is in each case independently selected from hydrogen, C₁₋₈ alkyl, aryl, C₁₋₈ heteroaryl, C₃₋₈ cycloalkyl, and C₁₋₈ heterocyclyl. In some embodiments, R^{2b} is selected from Cl, Br, C₁₋₆ alkoxy, and C(O)OH.

In some embodiments:
R^{2a} is selected from F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}, C(O)N(R^{z2*})₂, N(R^{2a*})C(O)R^{2a*}, OC(O)N(R^{2a*})₂, and N(R^{2a*})C(O)N(R^{z2*})₂, wherein R^{2a*} is in each case independently selected from hydrogen, C₁₋₈ alkyl, aryl, C₁₋₈ heteroaryl, C₃₋₈ cycloalkyl, and C₁₋₈ heterocyclyl; and
R^{2b} is selected from F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, and N(R^{2b*}) C(O)N(R^{2b*})₂, wherein R^{2b*} is in each case independently selected from hydrogen, C₁₋₈ alkyl, aryl, C₁₋₈ heteroaryl, C₃₋₈ cycloalkyl, and C₁₋₈ heterocyclyl.

In some embodiments:
R^{2a} is selected from Cl, Br, CN, C₁₋₆ alkoxy, NH₂, NH(C₁₋₆ alkyl), N(C₁₋₈ alkyl)₂, and C(O)OH; and
R^{2b} is selected from Cl, Br, CN, C₁₋₆ alkoxy, NH₂, NH(C₁₋₆ alkyl), N(C₁₋₈ alkyl)₂, and C(O)OH.

In some embodiments:
R^{2a} is selected from C₁₋₆ alkoxy; and
R^{2b} is selected from C₁₋₆ alkoxy and Cl.

In some embodiments, R^{2a} is H and R^{2b} is H. In some embodiments, R^{2a} is Cl and R^{2b} is C₁. In some embodiments, R^{2a} is F and R^{2b} is F. In some embodiments, R^{2a} is Br and R^{2b} is Br. In some embodiments, R^{2a} is C₁₋₆ alkoxy and R^{2b} is C₁₋₆ alkoxy. In some embodiments, R^{2a} is C(O)OH and R^{2b} is C(O)OH. In some embodiments, R^{2a} is C₁₋₆ alkoxy and R^{2b} is F, I, Cl, or Br. In some embodiments, R^{2a} is C(O)OH and R^{2b} is F, I, Cl, or Br. In some embodiments, R^{2a} is C₁ and R^{2b} is Br. In some embodiments, R^{2a} is C₁ and R^{2b} is F. In some embodiments, R^{2a} is C₁ and R^{2b} is I. In some embodiments, R^{2a} is Br and R^{2b} is F.

In some embodiments, R^{2a} is H, and R^{2b} is selected from F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, and N(R^{2b*}) C(O)N(R^{2b*})₂, wherein R^{2b*} is in each case independently selected from hydrogen, C₁₋₈ alkyl, aryl, C₁₋₈ heteroaryl, C₃₋₈ cycloalkyl, and C₁₋₈ heterocyclyl.

In some embodiments, R^{2a} is hydrogen; and R^{2b} is selected from Cl, Br, CN, C₁₋₆ alkoxy, NH₂, NH(C₁₋₆ alkyl), N(C₁₋₈ alkyl)₂, and C(O)OH. In some embodiments, R^{2a} is hydrogen; and R^{2b} is C₁, F, Br, or I. In some embodiments, R^{2a} is hydrogen; and R^{2b} is C₁₋₆ alkoxy. In some embodiments, R^{2a} is hydrogen; and R^{2b} is C(O)OH.

In some embodiments, R^{2a} is H, and R^{2b} is selected from Cl, Br, and C₁₋₆ alkoxy.

In some embodiments, R^{z1} is C₁₋₈ alkyl. In some embodiments, R^{z1} is C₁₋₆ alkyl. In some embodiments, R^{z1} is C₁₋₈ alkyl, substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H. In some embodiments, R^{z1} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H. In some embodiments, R^{z1} is C₁₋₈ alkyl, substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H. In some embodiments, R^{z1} is C₁₋₈ alkyl, substituted by N⁺(C₁₋₃ alkyl)₃. In some embodiments, R^{z1} is C₁₋₈ alkyl, substituted by SO₃H. In some embodiments, R^{z1} is C₁₋₈ alkyl, substituted by PO₃H₂. In some embodiments, R^{z1} is C₁₋₈ alkyl, substituted by CO₂H.

In some embodiments, R^{z3} is C₁₋₈ alkyl. In some embodiments, R^{z3} is C₁₋₆ alkyl. In some embodiments, R^{z3} is C₁₋₈ alkyl, substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H. In some embodiments, R^{z3} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H. In some embodiments, R^{z3} is C₁₋₈ alkyl, substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H. In some embodiments, R^{z3} is C₁₋₈ alkyl, substituted by N⁺(C₁₋₃ alkyl)₃. In some embodiments, R^{z3} is C₁₋₈ alkyl, substituted by SO₃H. In some embodiments, R^{z3} is C₁₋₈ alkyl, substituted by PO₃H₂. In some embodiments, R^{z3} is C₁₋₈ alkyl, substituted by CO₂H.

In some embodiments:
R^{z1} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H; and
R^{z3} is C₁₋₈ alkyl, substituted by NH₂, NHC₁₋₃ alkyl, N(C₁₋₃ alkyl)₂, N⁺(C₁₋₃ alkyl)₃, SO₃H, or PO₃H₂.

In some embodiments, R^{z1} is C₃ alkyl substituted with N′ (C₁₋₃ alkyl)₃. In some embodiments, R^{z3} is C₃ alkyl substituted with N⁺(C₁₋₃ alkyl)₃.

In some embodiments:
R^{z1} is C₁₋₈ alkyl;
R^{z3} is C₁₋₈ alkyl, substituted by NH₂, NHC₁₋₃ alkyl, N(C₁₋₃ alkyl)₂, N⁺(C₁₋₃ alkyl)₃, SO₃H, or PO₃H₂.

In some embodiments:
R^{z1} is C₁₋₈ alkyl; and
R^{z3} is C₁₋₈ alkyl, substituted by N⁺(C₁₋₃ alkyl)₃.

In some embodiments:
R^{z1} is C₁₋₈ alkyl, substituted by N⁺(C₁₋₃ alkyl)₃; and
R^{z3} is C₁₋₈ alkyl, substituted by N⁺(C₁₋₃ alkyl)₃.

In some embodiments:
R^{z1} is C₁₋₈ alkyl, substituted by SO₃H; and
R^{z3} is C₁₋₈ alkyl, substituted by N⁺(C₁₋₃ alkyl)₃.

In some embodiments:
R^{z1} is C₁₋₈ alkyl, substituted by SO₃H; and
R^{z3} is C₁₋₈ alkyl, substituted by SO₃H.

In some embodiments:
R^{z1} is C₁₋₈ alkyl, substituted by PO₃H₂; and
R^{z3} is C₁₋₈ alkyl, substituted by PO₃H₂.

In some embodiments:
R^{z1} is C₁₋₈ alkyl, substituted by CO₂H; and
R^{z3} is C₁₋₈ alkyl, substituted by CO₂H.

In some embodiments:
R^{z1} is C₁₋₈ alkyl, substituted by SO₃H; and
R^{z3} is C₁₋₈ alkyl, substituted by PO₃H₂.

In some embodiments:
R^{z1} is C₁₋₈ alkyl, substituted by CO₂H; and
R^{z3} is C₁₋₈ alkyl, substituted by N⁺(C₁₋₃ alkyl)₃.

In some embodiments, Z⁵ is CR^{a}R^{b}. In some embodiments, R^{a} is C(O)OR^{a1} and R^{b} is C(O)OR^{b1}. In some embodiments, R^{a} is CN and R^{b} is C(O)OR^{b1}.

In some embodiments, the compound of Formula (II) is selected from any one of the following compounds:

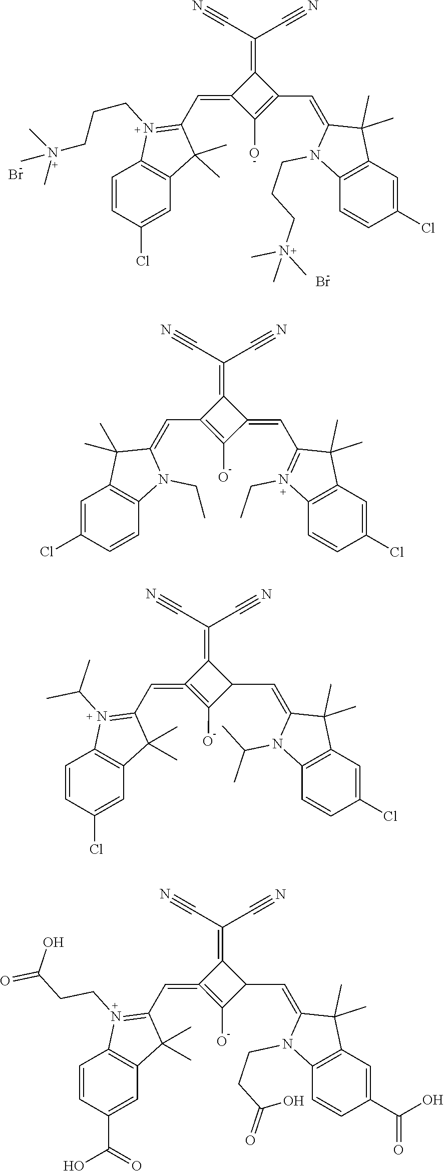

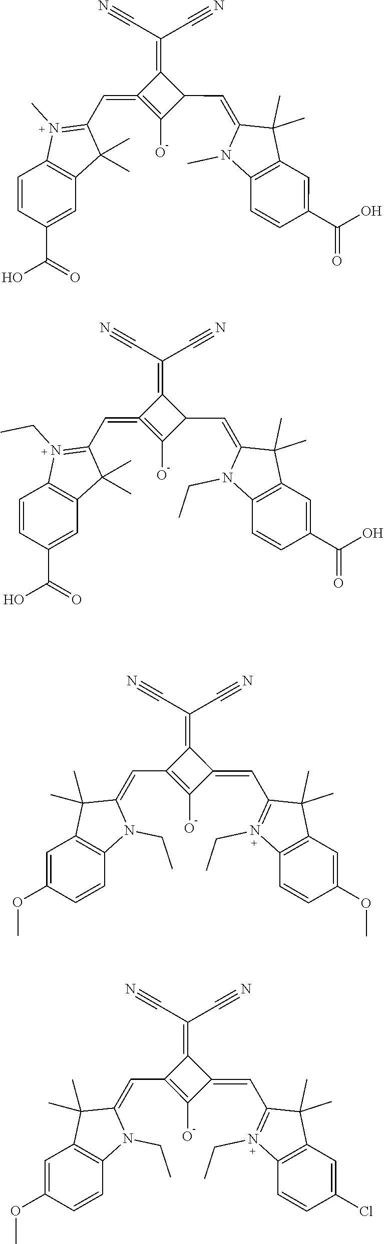

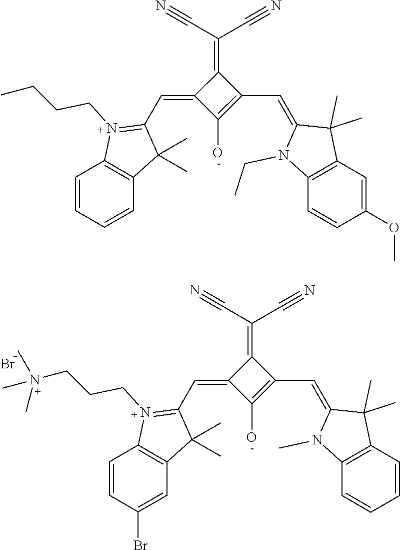

or a pharmaceutically acceptable salt thereof.

In some embodiments, the compound of Formula (II) is selected from any one of the following compounds:

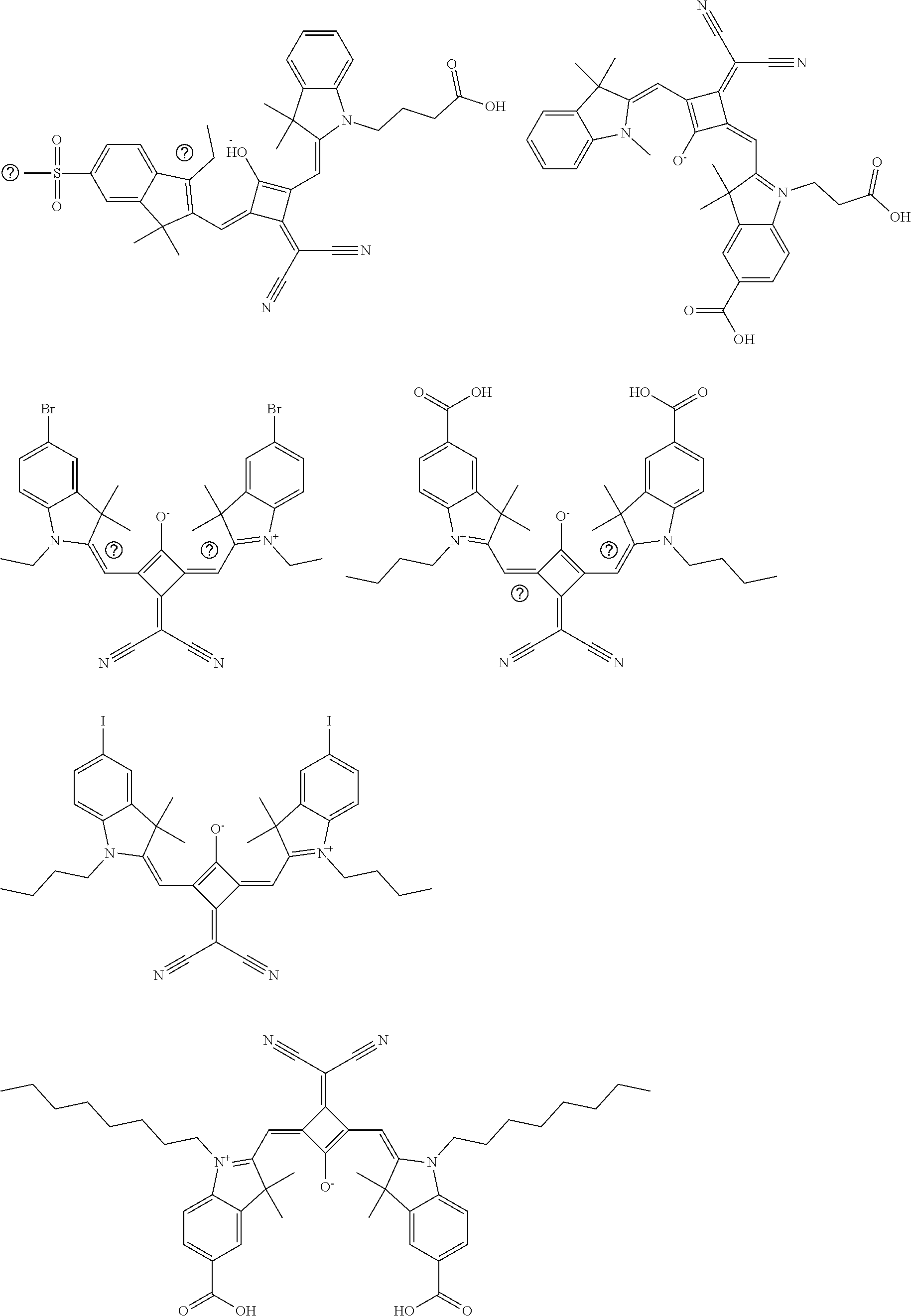

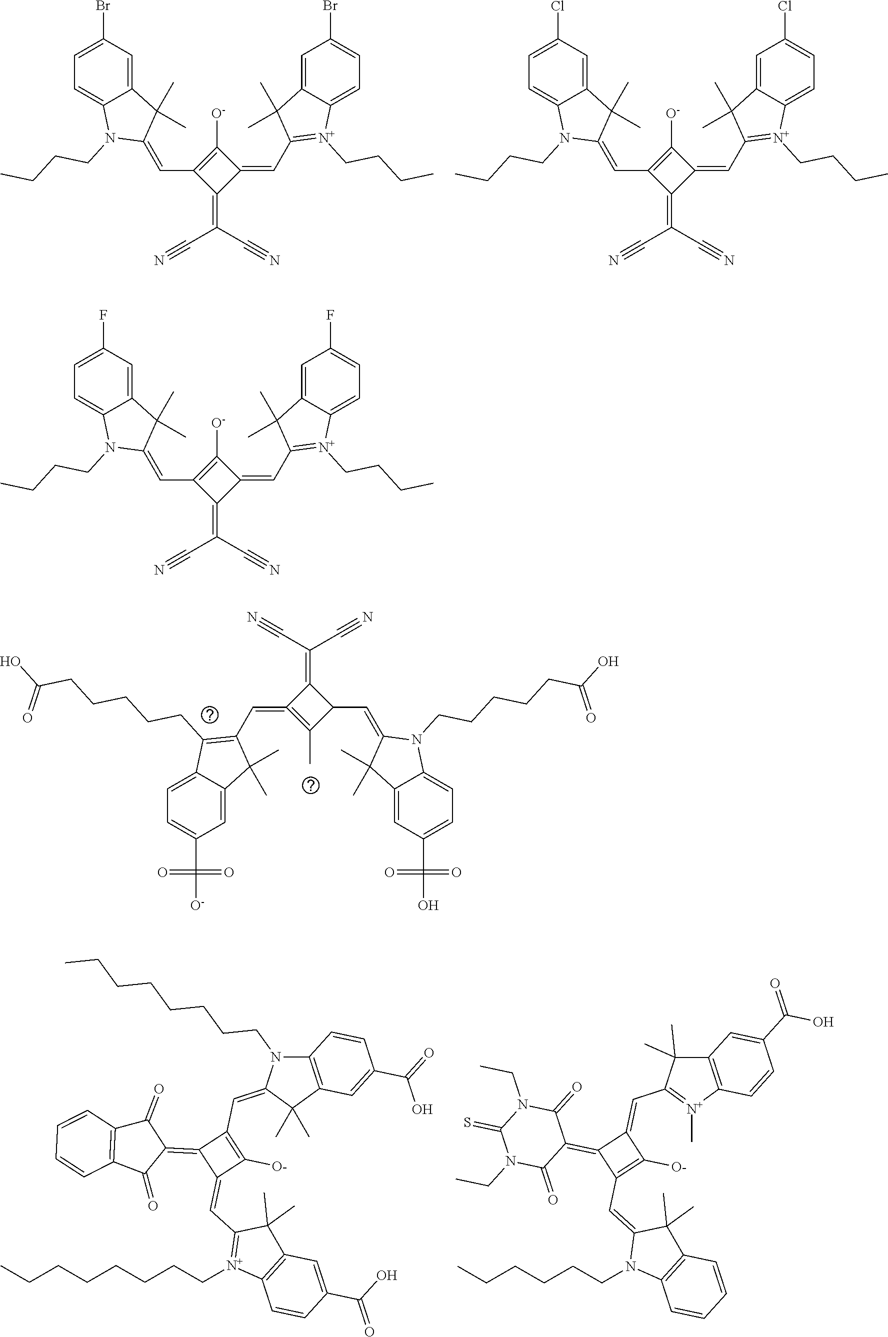

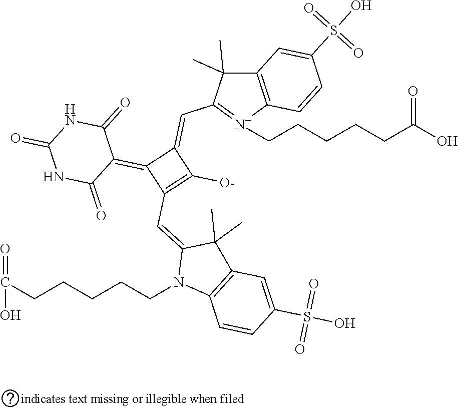 [SMILES: CC1(C)C(/C=C2C([O-])=C(/C=C3/N(CCCCCC(=O)O)c4ccc(S(=O)(=O)O)cc4C3(C)C)C\2=C2C(=O)NC(=O)NC2=O)=[N+](CCCCCC(=O)O)c2ccc(S(=O)(=O)O)cc21]

or a pharmaceutically acceptable salt thereof.

In some embodiments, the squaraine fluorophore of this disclosure is a compound selected from:

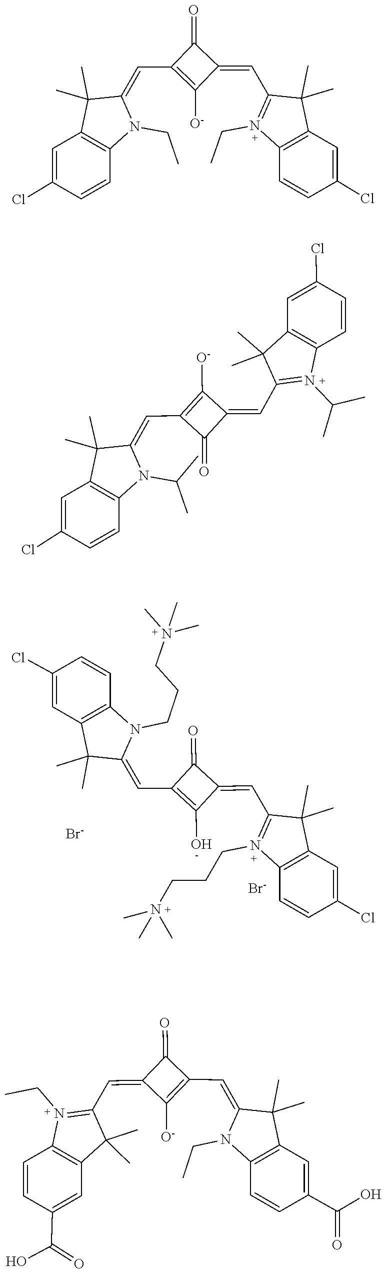

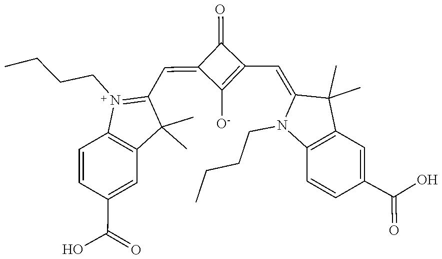

or a pharmaceutically acceptable salt thereof.

In some embodiments, the squaraine fluorophore of this disclosure is a compound selected from:

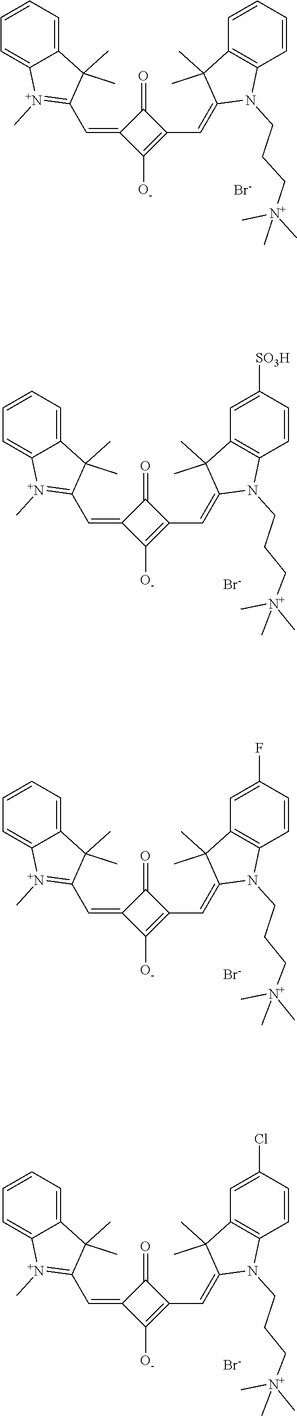

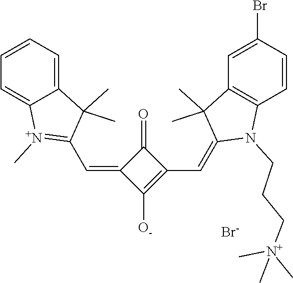

or a pharmaceutically acceptable salt thereof.

Without being bound by any particular theory or speculation, it is believed that the compounds of this disclosure possess fluorescent properties. In other words, the compounds are capable or emitting electromagnetic radiation or light of a wavelength. In some embodiments, the radiation is visible (e.g., visible light) or invisible (e.g., ultraviolet, infrared, or near-infrared radiation). In some embodiments, the compounds are capable to absorbing light or radiation of a short wavelength and emitting light or radiation of a longer wavelength. In some embodiments, the emission maximum for the compounds (e.g., of Formula (II) is within near-infrared or near-infrared II wavelength spectrum. In some embodiments, the emission maximum wavelength for the compound (e.g., of Formula (II)) is from about 600 nm to about 1800 nm, from about 600 to about 1000 nm, form about 650 to about 950 nm, from about 950 nm to about 1750 nm, from about 1000 to about 1700 nm, or from 1050 to about 1650 nm. In some embodiments, the emission maximum wavelength for the compound of Formula (I) is about 650 nm, about 700 nm, about 750 nm, about 800 nm, about 900 nm, about 950 nm, about 1000 nm, about 1050 nm, about 1100 nm, about 1150 nm, about 1200 nm, about 1250 nm, about 1300 nm, about 1500, about 1600 nm, about 1650 nm, about 1700 nm, or about 1750 nm. Without being bound by any particular theory of speculation, the emission maximum wavelength in NIR II window advantageously allows to use the compounds (e.g., of Formula (II)) for fluorescent imaging, because the compound's emitted NIR II radiation can be detected by a NIR-II surgical navigation system, a NIR-II camera, a NIR-II confocal/spinning-disc confocal microscope, a NIR-II light sheet microscope, NIR-II two-photon/multiphoton microscope, and NIR-II fluorescence lifetime microscope. In some embodiments, the compound (e.g., of Formula (II)) is water-soluble. For example, aqueous solubility of the compound (e.g., of Formula (II)) is from about 1 g/L to about 250 g/L, or about 5 g/L, about 10 g/L, about 15 g/L, about 20 g/L, about 25 g/L, or about 100 g/L.

### Pharmaceutically Acceptable Salts

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

### Methods of Use

In one general aspect, the present application relates to compounds described herein (e.g., of Formula (II)) useful in imaging techniques, diagnosing and monitoring treatment of various diseases and conditions described herein. In some embodiments, because the compounds can emit light after absorbing light, the compounds are useful in fluorescence imaging or optical imaging. In some embodiments, fluorescence imaging is NIR-II fluorescence imaging. In some embodiments, fluorescent imaging is carried out to detect light emitted by the compound at a wavelength from about 950 nm to about 1750 nm, from about 1000 nm to about 1700 nm, about 1000 nm, about 1100 nm, about 1200 nm, about 1300 nm, about 1400 nm, about 1500 nm, about 1600 nm, or about 1700 nm.

Fluorescence imaging is a type of non-invasive imaging technique that can help visualize biological processes taking place in a living organism. This imaging technique is very sensitive, allowing to detect fluorophore-containing compounds in biological tissues even at picomolar concentration. The method commonly includes administering exogenously a fluorophore compound to a patient, then exciting (e.g., irradiating) the compound by pointing a source of exciting radiation (e.g., light) to a tissue where the compound is expected to be accumulated, the then detecting fluorescent emission at the tissue site. In some embodiments, the irradiating comprises irradiating the compound at a wavelength of at least about 600 nm, at least about 650 nm, at least about 675 nm, at least about 700 nm, at least about 725 nm, at least about 750 nm, at least about 775 nm, at least about 800 nm, at least about 825 nm, at least about 850 nm, at least about 875 nm, at least about 900 nm, at least about 925 nm, at least about 950 nm, at least about 975 nm, or at least about 1,000 nm. In some embodiments, irradiating comprises irradiating the compound at a wavelength from 500-1,000 nm, 600-1,000 nm, 700-1,000 nm, 800-1,000 nm, 900-1,000 nm, 500-700 nm, 550-750 nm, 600-900 nm, 600-800 nm, 600-750 nm, 600-700 nm, or 650-750 nm.

In some embodiments, the present disclosure provides a method of imaging a tissue (e.g., cancerous tumor) in a subject, the method comprising (i) administering to the subject an effective amount of a compound of Formula (II), or a pharmaceutically acceptable salt thereof, or a pharmaceutical composition of comprising same, (ii) waiting a time sufficient to allow the compound to accumulate in the tissue (e.g., cancerous tumor) to be imaged; and (iii) imaging the tissue (e.g., cancerous tumor) with a fluorescence imaging technique. In some embodiments, the compound is administered systemically (e.g., using an injection and/or an injectable or infusible solution, or by an oral route, e.g., as described herein). In some embodiments, the tissue is selected from epithelial tissue, mucosal tissue, connective tissue, muscle tissue, skin tissue, fibrous tissue, vascular tissue, and nervous tissue. In some embodiments the is tissue is at or near an organ selected from lung, stomach, intestines, liver, thyroid, bladder, heart, eye, skin, kidney, gland, brain, pancreas, colon, lymph node, spleen, and prostate. In some embodiments, the issue is a cancerous tumor tissue. Exemplary cancers include acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary-cervical cancer, cholangiocarcinoma, chordoma, chronic lymphocytic leukemia (CLL), chronic myelogenous leukemia (CML), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney-langerhans cell histiocytosis, laryngeal cancer, laryngeal cancer and papillomatosis, leukemia, lip and oral cavity cancer, liver cancer (primary), lung cancer, lung cancer, lymphoma-macroglobulinemia, Waldenström-Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sézary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenström Macroglobulinemia, Wilms Tumor.

In some embodiments, the time sufficient to allow the compound of Formula (I) to accumulate in the cancerous tumor is from about 1 hour to about 168 hours, from about 2, about 3 or about 4 hours to about 168 hours, from about 48 hours to about 96 hours, about 24 hours, about 48 hours, about 72 hours, about 96 hours, about 120 hours, about 144 hours, or about 168 hours.

In some embodiments, the fluorescence imaging technique is NIR-II fluorescence imaging. The imaging can be carried out using a near infrared camera, imaging goggles, or telescope, or a similar device. In some embodiments, the device contains a source of light or irradiation to excite the fluorophore.

In some embodiments, the present disclosure provides a method of diagnosing (or early detection) of a disease or disorder (e.g., any of the cancers described herein). The method may include imaging the tissue (e.g., cancerous tissue) as described herein. For example, the method can include (i) administering to the subject an effective amount of a compound of Formula (II), or a pharmaceutically acceptable salt thereof, or a pharmaceutical composition comprising same; (ii) waiting a time sufficient to allow the compound to accumulate in the tissue (e.g., cancerous tumor), and (iii) imaging the tissue with an imaging technique.

In some embodiments, the present disclosure provides a method of treating cancer (any of the cancers described herein), the method comprising: (i) imaging a cancerous tumor in a subject according to an imaging method as described herein; and (ii) administering to the patient a therapeutically effective amount of an anti-cancer compound, or a pharmaceutically acceptable salt thereof. Suitable examples of anticancer agents include mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, anti-hormones, angiogenesis inhibitors, cytostatic agents anti-androgens, targeted antibodies, HMG-COA reductase inhibitors, and prenyl-protein transferase inhibitors. Other exemplary anti-cancer agents include nucleoside analogues, antifolates, antimetabolites, topoisomerase I inhibitor, anthracyclines, podophyllotoxins, taxanes, vinca alkaloids, alkylating agents, platinum compounds, proteasome inhibitors, nitrogen mustards & oestrogen analogue, monoclonal antibodies, tyrosine kinase inhibitors, mTOR inhibitors, retinoids, immunomodulatory agents, histone deacetylase inhibitors, and combinations thereof. In certain embodiments, the anti-cancer agent is selected from one or more of abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase Erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, recombinant hpv quadrivalent vaccine, recombinant hpv nonavalent vaccine, obinutuzumab, gefitinib, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, and pharmaceutically acceptable salts thereof. Furthermore, the disclosed compounds (in addition to being imaging agents) can have cytotoxic properties and as such may be effectively used for the treatment of proliferative disorders, including cancer and similar diseases as described herein. In the field of medical oncology it is normal practice to use a combination of different forms of treatment to treat each patient with cancer. In medical oncology the other component(s) of such conjoint treatment in addition to compositions of the present invention may be, for example, surgery, radiotherapy, chemotherapy, signal transduction inhibitors and/or monoclonoal antibodies.

In some embodiments, the present disclosure provides a method of treating cancer (any of the cancers described herein), the method comprising: (i) imaging a cancerous tumor in a subject according to an imaging method as described herein; and (ii) surgically removing the cancerous tumor from the subject. In some aspects of these embodiments, the present disclosure provides a method for in intraoperative optical and/or fluorescence imaging and image-guided cancer surgery. The imaging can be carried out as described herein, e.g., using a near infrared camera or vision goggles. Before imaging, the method may include administering a cancer-targeting fluorophore of this disclosure (e.g., a compound of Formula (II)) and then waiting a sufficient amount of time (e.g., 24 hours, 48 hours, 96 hour, or more) for the cancer-targeting compound to accumulate in the cancerous tissue. Suitable examples of cancer surgeries include staging surgery, tumor removal, debulking surgery, palliative surgery, reconstructive surgery, preventive surgery, laparoscopic surgery, laser surgery, cryosurgery, Mohs surgery, and endoscopy.

In some embodiments, the present disclosure provides a method of monitoring treatment of cancer in a subject, the method comprising (i) administering to the subject an effective amount of a compound of this disclosure (e.g., compound of Formula (II)), or a pharmaceutically acceptable salt thereof, or a pharmaceutical composition comprising same, (ii) waiting a time sufficient to allow the compound of Formula (II) to accumulate in cancerous tumor of the subject; (iii) imaging the cancerous tumor of the subject with an imaging technique; and (iv) administering to the subject a therapeutic agent in an effective amount to treat the cancer. In some embodiments, the method further includes step (v) after (iv), administering to the subject an effective amount of a compound this disclosure (e.g., a compound of Formula (II)), or a pharmaceutically acceptable salt thereof, or a pharmaceutical composition comprising same; (vi) waiting a time sufficient to allow the compound of this disclosure (e.g., a compound of Formula (II)) to accumulate in the cancerous tumor of the subject; (vii) imaging the cancerous tumor of the subject with an imaging technique; and (viii) comparing the image of step (iii) and the image of step (vii). In one example, comparing the images is indicative of successful treatment of the cancer. Suitable examples of therapeutic agents useful to treat cancer include those described herein. In one general aspect, the present disclosure also provides a method of treating cancer in a subject in need thereof, comprising administering to the subject a squaraine fluorophore of this disclosure, or a pharmaceutically acceptable salt thereof.

The present disclosure further provides a pharmaceutical composition, which comprises a compound of this disclosure (e.g., a compound of Formula II or a pharmaceutically acceptable salt thereof), as described herein. In one embodiment, the pharmaceutical composition includes the disclosed compounds together with a pharmaceutically acceptable diluent or carrier. The present disclosure further provides a disclosed compound or a pharmaceutically acceptable salt thereof, for use in therapy. In one embodiment, the invention provides the disclosed compounds or a pharmaceutically acceptable salt thereof, for use in the treatment of cancer in a mammal. In one embodiment, the cancer is neuroblastoma. In one embodiment, the cancer is ovarian cancer. In one embodiment, the cancer is pancreatic cancer. In one embodiment, the cancer is colorectal cancer. In one embodiment, the cancer is prostate cancer. Another embodiment of the present disclosure provides the use of the disclosed compounds, or a pharmaceutically acceptable salt thereof, in the manufacture of a medicament for the treatment of cancer in a mammal.

### Compositions, Formulations, and Routes of Administration

The present application also provides pharmaceutical compositions comprising an effective amount of a compound of the present disclosure disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. The composition can be administered by any route by which a fluorophore is effectively administrable and that facilitates imaging of a tumorous tissue. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

The compounds disclosed herein may be formulated in a wide variety of pharmaceutical compositions for administration to a patient. Such compositions include, but are not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

In some embodiments, the compounds disclosed herein may be formulated as nanoparticles. The nanoparticles may have an average particle size from 1-1,000 nm, preferably 10-500 nm, and even more preferably from 10-200 nm.

### Dosages and Regimens

In the pharmaceutical compositions of the present application, a compound of the present disclosure is present in an effective amount (e.g., a therapeutically effective amount). Effective doses of the imageable compounds may vary, depending on the diseases treated, the severity of the disease, the route of administration, the sex, age and general health condition of the subject, excipient usage, the possibility of co-usage with other imaging agents or therapeutic treatments such as use of other agents and the judgment of the treating physician, lab technician, or a diagnostician.

In some embodiments, an effective amount of the compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered as needed for imaging, for example, on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes kits useful, for example, in the imaging and/or treatment of disorders, diseases and conditions referred to herein, which include one or more containers containing a pharmaceutical composition comprising an effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit.

### Definitions

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). Unless specified to the contrary, an “alkyl” group includes both saturated alkyl groups and unsaturated alkyl groups. A saturated alkyl group does not include any carbon-carbon double bonds or carbon-carbon triple bonds. An unsaturated alkyl group contains at least one double or triple carbon-carbon bond. In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ saturated alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkylenyl” refers to a divalent radical of a straight-chain, cyclic, or branched saturated hydrocarbon group having a specified range of carbon atoms (e.g., a “C₁₋₁₆ alkyl” can have from 1 to 16 carbon atoms). An example of alkylenyl is a methylene (—CH₂-). An alkylenyl can be substituted as described above for an alkyl.

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁-6 haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include-CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended to the parent molecular moiety through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and/or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (₅ₛ), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

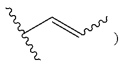

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”).

In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂-+alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C_{2_4} alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofurany 1, tetrahydrothiopheny 1, dihydrothiopheny 1, pyrrolidiny 1, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b] pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5, 7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb}) R^{aa}, —C(═NR^{bb}) OR^{aa}, —OC(═NR^{bb}) R^{aa}, —OC(═NR^{bb}) OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{a}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc}) OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{cc})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{cc}, —OC(═NR^{ff}) Rec, —OC(═NR^{ff}) OR^{cc}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R&& groups, or two geminal R^{dd} substituents can be joined to form ═O or ═S; wherein X-is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂ (C₁₋₆ alkyl)⁺X⁻, —NH₃⁺X⁻, —N(OC₁₋₆ alkyl) (C₁₋₆ alkyl), —N(OH) (C₁₋₆ alkyl), —NH(OH), —SH, —SC₁₋₆ alkyl, —SS(C₁₋₆ alkyl), —C(═O) (C₁₋₆ alkyl), —CO₂H, —CO₂ (C₁₋₆ alkyl), —OC(═O) (C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O) (C₁₋₆ alkyl), —N(C₁₋₆ alkyl) C(═O) (C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂ (C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S) N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O) (C₁₋₆ alkyl)₂, —OP(═O) (C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb}) R^{aa}, —OC(═NR^{bb}) OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR)₂, —OP(OR)₃⁺X⁻, —OP(═O)(R^{aa})₂, —OP(═O)(OR^{cc})₂, and —OP(═O)(N(R^{bb})₂)₂, wherein X⁻, R^{aa}, R^{bb} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₂ and —N(R^{bb})₃⁺X⁻, wherein R^{bb} and X⁻ are as defined herein.

The term “sulfonyl” refers to a group selected from-SO₂N(R^{bb})₂, —SO₂R^{aa}, and SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S (R^{X1}), —C(═NR^{X1})R^{X1}, —C(═NR^{X1})OR^{X1}, —C(═NR^{X1}) SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, butare not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb}) R^{aa}, —C(═NR^{bb}) OR^{aa}), C(═NR^{bb})N(R^{bb})₂, wherein R^{aa} and R^{bb} are as defined herein.

The term “oxo” refers to the group —O, and the term “thiooxo” refers to the group ═S.

The term “cyano” refers to the group —CN.

The term “azide” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb}) R^{aa}, —C(═NR^{cc}) OR^{aa}, —C(═NR^{cc})N(R^{cc})₂, —SO₂N(RC)₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

As used herein, a chemical bond depicted: [symbol] represents either a single, double, or triple bond, valency permitting. By way of example,

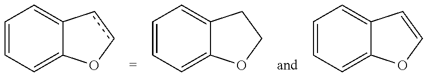

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}, C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Unless stated to the contrary, a substituent drawn without explicitly specifying the point of attachment indicates that the substituent may be attached at any possible atom. For example, in a benzofuran depicted as:

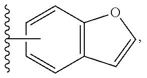

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

As used herein, the term “mammal” refers to a warm-blooded animal that has or is at risk of developing a disease described herein and includes, but is not limited to, guinea pigs, dogs, cats, rats, mice, hamsters, and primates, including humans.

As used interchangeably herein, “subject,” “individual,” or “patient,” refers to a vertebrate, preferably a mammal, more preferably a human. Mammals include, but are not limited to, murine, simians, humans, farm animals, sport animals, and pets. The term “pet” includes a dog, cat, guinea pig, mouse, rat, rabbit, ferret, and the like.

The term farm animal includes a horse, sheep, goat, chicken, pig, cow, donkey, llama, alpaca, turkey, and the like.

As used herein, “administration” refers to the injection of active agent on the subject. Exemplary methods of administration include: intravenously (i.v.), intraperitoneally (i.p.), intratumorally (i.t.), or subcutaneously (s.c.) such as tissue ipsilateral (i.l.) to the tumor and tissue contralateral (c.l.) to the tumor.

As used herein, “control” is an alternative subject or sample used in an experiment for comparison purposes and included to minimize or distinguish the effect of variables other than an independent variable. A “control” can be positive or negative.

As used herein, “therapeutic” generally refers to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Example 1—Preparation of Exemplified Compounds

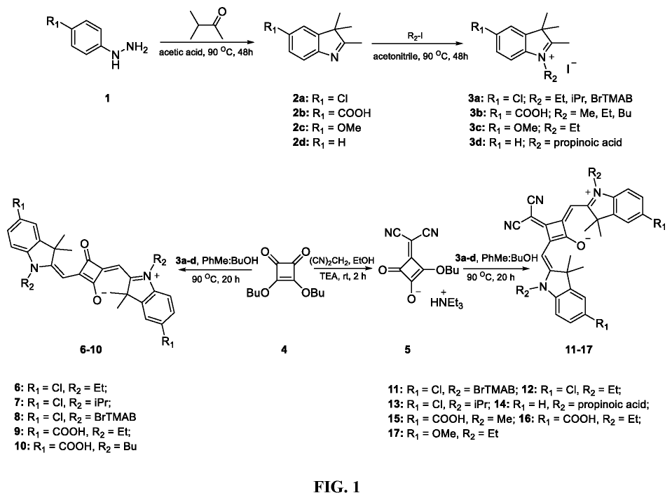

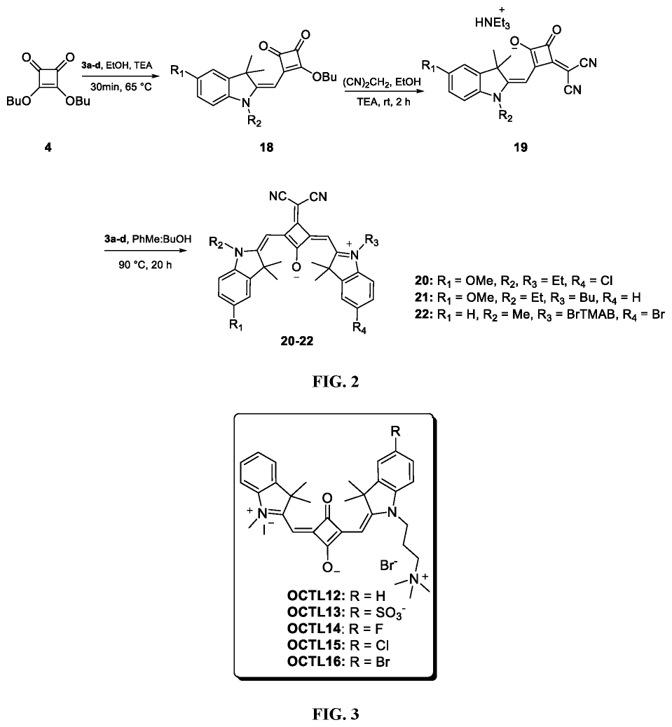

Synthetic schemes showing preparation of exemplified compounds are provided in FIG. 1 (synthesis of symmetrical squaraine dyes 6-17) and FIG. 2 (synthesis of asymmetrical squaraine dyes 20-22). Referring to FIG. 1, synthesis of symmetrical squaraine dyes 6-17 begins with the Fischer indole synthesis of the heterocyclic aromatic indole compounds 3a-d. Compounds 3a-d were synthesized with similar synthetic procedures reported previously. The synthesis of salts 3a-d first involved the reaction of the substituted phenyl hydrazine 1 with 3-methyl-2-butanone in acetic acid under reflux for 48 hrs. The reaction progress was monitored with thin layer chromatography (TLC), and upon reaction completion, excess solvent was removed under reduced pressure and the reaction mixture was washed with saturated sodium bicarbonate. Furthermore, the aromatic heterocyclic compound 2a-d was reacted with the corresponding alkylating agent under reflux in acetonitrile for 48 hrs. Reaction progress was monitored using TLC, and upon completion, excess solvent was removed under reduced pressure and final salt compounds 3a-d were precipitated from dichloromethane and diethyl ether. The alkylated heterocyclic compounds 3a-d were then reacted with dibutyl squarate 4 in a 1:1 azeotropic solvent mixture of toluene/butanol to yield oxycyclobutenolate symmetrical squaraine dyes 6-10 in good yields ranging from 55-70%. A similar reaction procedure is utilized to obtain the symmetrical dicyanomethylene substituted squaraine dyes 11-17, which first involved functionalizing the dibutyl squarate 4 with the dicyanomethylene group using malononitrile and ethanol as a solvent to yield compound 5. Referring to FIG. 1, synthesis of asymmetrical squaraine dyes 20-22 first involved the formation of the semi-squaraine 18, by the condensation reaction of one equivalent of both the alkylated heterocyclic compound 3a-d and dibutyl squarate 4 under basic conditions. Analogous to Scheme 1, upon further reaction with malononitrile under basic conditions, the compound 18 is functionalized with the dicyanomethylene group to obtain compound 19. The final asymmetrical compounds 20-22 were obtained from a reaction of compound 19 with a second heterocyclic equivalent of 3a-d in decent yields ranging from 32-65%.

### General Procedure for the Synthesis of Indolium Salts 3a-d:

3-methyl-2-butanone (1.5 mol eq.) and the respective substituted phenyl hydrazine (1 mol eq.) were refluxed in acetic acid for 48 hrs. The reaction progress was monitored with TLC and at reaction completion, the solution was cooled, and excess solvent was removed under reduced pressure. The reaction mixture was washed with a saturated bicarbonate solution and the organic layer was collected using dichloromethane. The heterocycle 2a-d (1 mol eq.) was refluxed for 48 hours in acetonitrile with the respective alkylating agent (3 mol eq.), and upon completion of reaction, excess solvent was removed under reduced pressure. The final compound was obtained through recrystallization with dichloromethane and ether. As stated earlier, the synthesis of these indolium salts were prepared using procedures reported by our group and others.

### General Procedure for the Synthesis of Symmetrical Oxycyclobutenolate Squaraines:

2,3,3-trimethylindolenine (2 mol eq) and 3,4-dihydroxycyclobut-3-ene-1,2-dione (1 mol eq) were refluxed in an azeotropic solvent mixture of n-butanol and toluene (1:1, 40 mL) for 20 hrs. The reaction progress was monitored with TLC and UV-vis spectrometer until reaction completion. Excess solvent was removed under reduced pressure, and the crude product was precipitated with methanol, filtered off and dried under vacuum. The precipitation procedure was repeated severally to obtain the pure product.

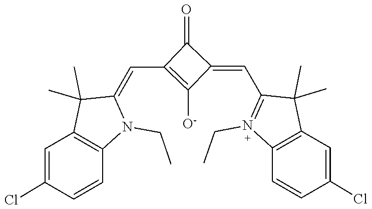

(E)-4-((5-chloro-1-ethyl-3,3-dimethyl-3H-indol-1-ium-2-yl)methylene)-2-(((Z)-5-chloro-1-ethyl-3,3-dimethylindolin-2-ylidene) methyl)-3-oxocyclobut-1-en-1-olate (6). It is obtained in 65% yield; ¹H NMR (400 MHZ, DMSO) δ 7.681 (s, 1H), 7.394 (m, 2H), 5.786 (s, 1H), 4.123 (m, 2H), 1.683 (s, 6H), 1.262 (m, 3H); ¹³C NMR (100 MHz, DMSO) § 172.30, 170.91, 166.40, 164.91, 144.05, 140.13, 129.06, 128.13, 122.84, 118.21, 112.37, 88.18, 49.29, 48.60, 25.66, 11.86. ESI-MS (positive mode) calculated for C₃₀H₃₀Cl₂N₂O₂: m/z 521.48, found: m/z 521.1815.

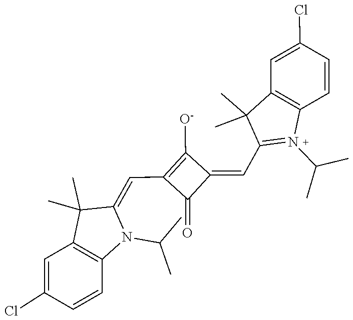

(E)-4-((5-chloro-1-isopropyl-3,3-dimethyl-3H-indol-1-ium-2-yl)methylene)-2-(((Z)-5-chloro-1-isopropyl-3,3-dimethylindolin-2-ylidene) methyl)-3-oxocyclobut-1-en-1-olate (7). It was obtained in 52% yield. ¹H NMR (400 MHZ, DMSO) δ 7.674 (d, J=2 Hz, 1H), 7.463 (d, J=8.4 Hz, 1H), 7.351 (dd, J=10.4 Hz, 1H), 5.917 (s, 1H), 4.879 (m, 1H), 1.663 (s, 6H), 1.542 (s, 6H); ¹³C NMR (100 MHz, DMSO) δ 181.09, 179.65, 169.56, 144.85, 140.26, 128.24, 127.97, 123.30, 114.02, 87.56, 49.20, 48.07, 26.86, 19.24. ESI-MS (positive mode) calculated for C₃₂H₃₄Cl₂N₂O₂: m/z 549.54, found: m/z 549.2130.

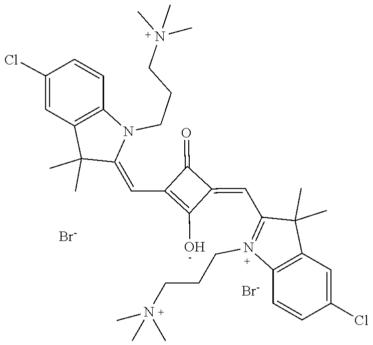

(E)-4-((5-chloro-3,3-dimethyl-1-(3-(trimethylammonio) propyl)-3H-indol-1-ium-2-yl)methylene)-2-(((Z)-5-chloro-3,3-dimethyl-1-(3-(trimethylammonio) propyl) indolin-2-ylidene) methyl)-3-oxocyclobut-1-en-1-olate bromide (8). It was obtained in 65% yield. ¹H NMR (400 MHZ, DMSO) § 7.699 (s, 1H), 7.449 (m, 2H), 5.863 (s, 1H), 4.144 (m, 2H, J=8.2 Hz), 3.454 (m, 2H, J=Hz), 3.097 (s, 9H), 2.130 (m, 2H, J=Hz), 1.708 (s, 6H); ¹³C NMR (100 MHz, DMSO) δ 180.97, 180.60, 143.92, 141.34, 128.67, 128.34, 123.29, 112.34, 87.28, 64.09, 62.90, 60.81, 58.12, 26.81, 20.93, 19.10, 17.61, 14.33.

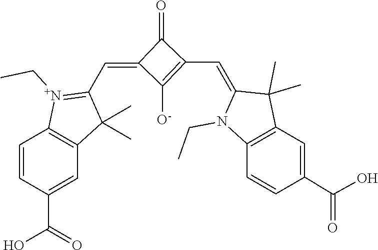 [SMILES: CCN1/C(=C\C2=C([O-])C(=C\C3=[N+](CC)c4ccc(C(=O)O)cc4C3(C)C)/C2=O)C(C)(C)c2cc(C(=O)O)ccc21]

(E)-4-((5-carboxy-1-ethyl-3,3-dimethyl-3H-indol-1-ium-2-yl)methylene)-2-(((Z)-5-carboxy-1-ethyl-3,3-dimethylindolin-2-ylidene) methyl)-3-oxocyclobut-1-en-1-olate (9). It was obtained in 57% yield. ¹H NMR (400 MHZ, DMSO) δ 8.037 (d, 1H, J=7.2 Hz), 7.962 (d, 1H, J=7.2 Hz), 7.425 (d, 1H, J=8 Hz), 5.888 (s, 1H), 4.161 (m, 1H), 1.709 (s, 6H), 1.294 (t, 3H, J=14 Hz); ¹³C NMR (100 MHZ, DMSO) δ 181.03, 169.83, 167.52, 145.89, 142.12, 130.77, 126.32, 123.68, 110.44, 87.68, 49.02, 26.81, 12.23

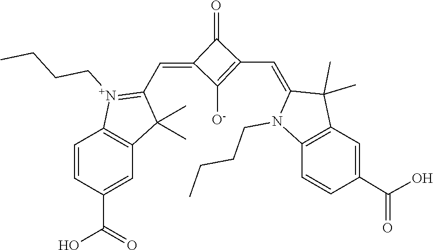 [SMILES: CCCCN1/C(=C\C2=C([O-])C(=C\C3=[N+](CCCC)c4ccc(C(=O)O)cc4C3(C)C)/C2=O)C(C)(C)c2cc(C(=O)O)ccc21]

(E)-4-((1-butyl-5-carboxy-3,3-dimethyl-3H-indol-1-ium-2-yl)methylene)-2-(((Z)-1-butyl-5-carboxy-3,3-dimethylindolin-2-ylidene) methyl)-3-oxocyclobut-1-en-1-olate (10). It was obtained in 60% yield. ¹H NMR (400 MHZ, DMSO) δ 12.867 (s, 1H), 8.038 (d, 1H, J=7.2 Hz), 7.972 (dd, 1H, J=7.8 Hz), 7.430 (d, 1H, J=7.2 Hz), 5.899 (s, 1H), 4.124 (m, 2H), 1.705 (s, 6H), 1.411 (m, 2H), 0.946 (t, 3H, J=14 Hz).

General Procedure for the Synthesis of Symmetrical dicyanomethylene Squaraines:

4-(dicyanomethylene)-2-butoxy-3-oxycyclobut-1-enolate (1 mol eq.) and 2,3,3-trimethylindolenine (2 mol eq.) were stirred and refluxed in an azeotropic solvent mixture of n-butanol and toluene (1:1, 40 mL) for 20 hrs. The reaction progress was monitored with UV-vis spectrometer and TLC. Upon reaction completion, excess solvent was removed under reduced pressure, and the final compound was precipitated out of methanol.

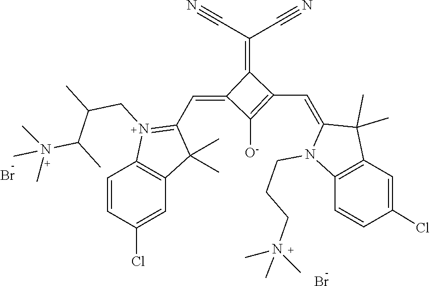

(Z)-4-((5-chloro-3,3-dimethyl-1-(3-(trimethylammonio) propyl)-3H-indol-1-ium-2-yl)methylene)-2-(((Z)-5-chloro-3,3-dimethyl-1-(3-(trimethylammonio) propyl) indolin-2-ylidene) methyl)-3-(dicyanomethylene) cyclobut-1-en-1-olate bromide (11). It is obtained in 64% yield; ¹H NMR (400 MHZ, DMSO) δ 7.761 (d, J=1.6 Hz, 1H), 7.591 (d, J=8.8 Hz, 1H), 7.519 (dd, J=10.4 Hz, 1H), 6.286 (s, 1H), 4.060 (t, J=14.8 Hz, 2H), 3.471 (m, 2H), 3.092 (s, 9H), 2.164 (m, 2H), 1.722 (s, 6H); ¹³C NMR (100 MHz, DMSO) δ 172.72, 171.93, 167.08, 165.92, 144.27, 140.76, 129.86, 128.59, 123.38, 119.08, 113.19, 89.08, 62.84, 58.14, 52.73, 49.88, 26.21, 20.98. ESI-MS (positive mode) calculated for C₄₁H₅₀N₆Cl₂Br₂O: m/z 873.60, found: m/z 743.4528.

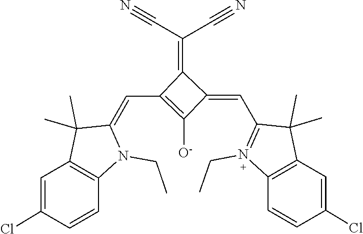

(Z)-4-((5-chloro-1-ethyl-3,3-dimethyl-3H-indol-1-ium-2-yl)methylene)-2-(((Z)-5-chloro-1-ethyl-3,3-dimethylindolin-2-ylidene) methyl)-3-(dicyanomethylene) cyclobut-1-en-1-olate (12). It was obtained in 60% yield. 1H NMR (400 MHZ, CDCl₃) δ 7.329 (d, J=5.2 Hz, 2H), 6.987 (d, J=8.8 Hz, 1H), 6.497 (s, 1H), 4.057 (m, 2H), 1.770 (s, 6H), 1.417 (t, J=14.4 Hz, 3H); ¹³C NMR (100 MHz, DMSO) § 172.76, 171.38, 166.86, 165.37, 144.51, 140.60, 129.52, 128.60, 123.31, 118.67, 112.84, 88.64, 49.75, 26.13, 12.33. ESI-MS (positive mode) calculated for C₃₃H₃₀N₄Cl₂O: m/z 569.53, found: m/z 569.1920.

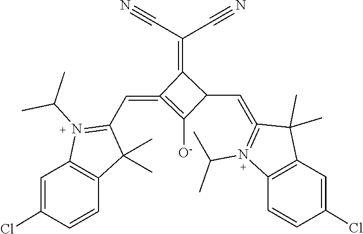

(Z)-4-((5-chloro-1-isopropyl-3,3-dimethyl-3H-indol-1-ium-2-yl)methylene)-2-(((Z)-5-chloro-1-isopropyl-3,3-dimethylindolin-2-ylidene) methyl)-3-(dicyanomethylene) cyclobut-1-en-1-olate (13). It was obtained in 55% yield. ¹H NMR (400 MHZ, DMSO) δ 7.705 (d, J=8.4 Hz, 1H), 7.565 (s, 1H), 7.418 (d, J=8.4 Hz, 1H), 6.335 (s, 1H), 4.813 (m, 1H), 1.681 (s, 6H), 1.575 (s, 6H); ¹³C NMR (100 MHz, DMSO) § 172.78, 172.07, 166.88, 165.05, 145.17, 140.26, 129.38, 128.26, 123.26, 119.01, 114.41, 89.90, 49.77, 49.20, 26.29, 19.23. ESI-MS (positive mode) calculated for C₃₅H₃₄N₄Cl₂O: m/z 597.58, found: m/z 597.2236

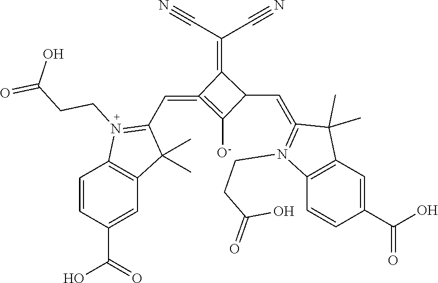

(Z)-4-((5-carboxy-1-(2-carboxyethyl)-3,3-dimethyl-3H-indol-1-ium-2-yl)methylene)-2-(((Z)-5-carboxy-1-(2-carboxyethyl)-3,3-dimethylindolin-2-ylidene) methyl)-3-(dicyanomethylene) cyclobut-1-en-1-olate (14). It was obtained in 56% yield. ¹H NMR (400 MHZ, DMSO) δ 7.541 (d, J=7.6 Hz, 1H), 7.392 (m, 2H), 7.251 (m, 1H), 6.267 (s, 1H), 3.923 (t, J=13.2 Hz, 2H), 2.808 (t, J=14 Hz, 2H) 1.677 (s, 6H); ¹³C NMR (100 MHz, DMSO) δ 172.84, 172.13, 170.55, 166.81, 165.38, 142.10, 128.54, 125.26, 122.73, 118.76, 111.83, 88.82, 64.58, 49.59, 31.75, 30.40, 26.37, 18.97, 13.98. ESI-MS (positive mode) calculated for C₃₅H₃₂N₄O₅: m/z 588.66, found: m/z 659.3295

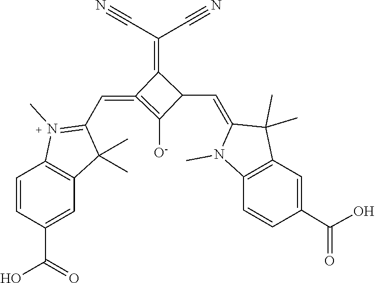

(Z)-4-((5-carboxy-1,3,3-trimethyl-3H-indol-1-ium-2-yl)methylene)-2-(((Z)-5-carboxy-1,3,3-trimethylindolin-2-ylidene) methyl)-3-(dicyanomethylene) cyclobut-1-en-1-olate (15). It was obtained in 62% yield. ¹H NMR (400 MHZ, DMSO) δ 8.065 (s, 1H), 8.014 (d, 1H, J-7.2 Hz), 7.508 (d, 1H, J=7.8 Hz), 6.378 (s, 1H), 3.618 (s, 3H), 1.710 (s, 6H); ¹³C NMR (100 MHz, DMSO) δ 173.52, 172.72, 167.42, 167.21, 166.19, 146.46, 142.33, 130.70, 127.26, 123.57, 118.67, 111.47, 89.97, 49.36, 32.01, 26.24

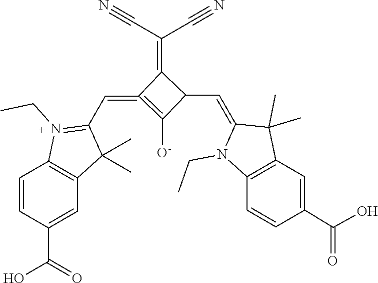

(Z)-4-((5-carboxy-1-ethyl-3,3-dimethyl-3H-indol-1-ium-2-yl)methylene)-2-(((Z)-5-carboxy-1-ethyl-3,3-dimethylindolin-2-ylidene) methyl)-3-(dicyanomethylene) cyclobut-1-en-1-olate (16: BNA-9). It was obtained in 55% yield . . . ¹H NMR (400 MHz, DMSO) § 8.084 (s, 1H), 7.999 (d, 1H, J=2 Hz), 7.517 (d, 1H, J=8.4 Hz), 6.402 (s, 1H), 4.122 (m, 2H), 1.728 (s, 6H), 1.302 (t, 3H, J=14 Hz); ¹³C NMR (100 MHz, DMSO) δ 172.60, 172.46, 167.40, 167.17, 166.21, 145.28, 142.63, 130.83, 127.34, 123.75, 118.53, 111.27, 89.57, 49.45, 26.24, 12.41

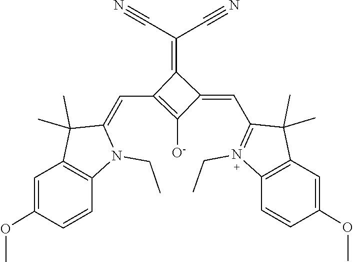

(Z)-3-(dicyanomethylene)-4-((1-ethyl-5-methoxy-3,3-dimethyl-3H-indol-1-ium-2-yl)methylene)-2-(((Z)-1-ethyl-5-methoxy-3,3-dimethylindolin-2-ylidene) methyl) cyclobut-1-en-1-olate (17). It is obtained in 70% yield; ¹H NMR (400 MHz, DMSO) § 7.325 (d, J=8.4 Hz, 1H), 7.206 (s, 1H), 6.968 (d, J=7.6 Hz, 1H), 6.222 (s, 1H), 4.063 (m, 2H), 3.806 (s, 3H), 1.680 (s, 6H), 1.268 (t, J=12.4 Hz, 3H); ¹³C NMR (100 MHz, DMSO) δ 173.21, 170.48, 166.42, 163.62, 158.03, 144.15, 135.21, 119.04, 113.63, 111.95, 109.58, 87.59, 56.29, 49.69, 26.33, 12.40. ESI-MS (positive mode) calculated for C₃₅H₃₆N₄O₃: m/z 560.70, found: m/z 560.2845

### General Procedure for the Synthesis of Asymmetrical Dicyanomethylene Squaraines:

The functionalized dicyanomethylene semi-squaraine (1 mol eq) and the corresponding indolenine salts were refluxed in an azeotropic solvent mixture of toluene and n-butanol (1:1, 40 mL) for 20 hrs. The reaction progress was monitored with UV-vis spectrometer and TLC. Upon reaction completion, excess solvent was removed under reduced pressure, and the final compound was purified by column chromatography with DCM: MeOH (95:5).

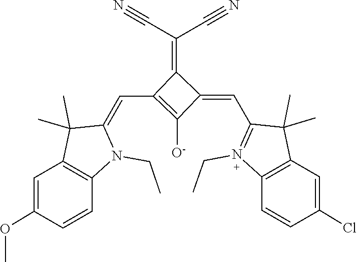

(Z)-4-((5-chloro-1-ethyl-3,3-dimethyl-3H-indol-1-ium-2-yl)methylene)-3-(dicyanomethylene)-2-(((Z)-1-ethyl-5-methoxy-3,3-dimethylindolin-2-ylidene) methyl) cyclobut-1-en-1-olate (20). It was obtained in 45% yield. ¹H NMR (400 MHZ, DMSO) δ 7.654 (s, 1H), 7.437 (m, 2H), 7.346 (d, J=8.4 Hz, 1H), 7.255 (s, 1H), 7.011 (d, J=8.4 Hz, 1H), 6.338 (s, 1H), 6.187 (s, 1H), 4.119 (m, 2H), 3.989 (m, 2H), 3.821 (s, 3H), 1.696 (s, 12H), 1.277 (m, 6H); ¹³C NMR (100 MHz, DMSO) δ 173.03, 172.37, 168.91, 166.69, 165.71, 162.54, 158.70, 144.70, 144.05, 140.91, 134.88, 128.43, 123.17, 118.87, 113.93, 112.91, 111.94, 109.50, 88.97, 87.50, 56.35, 50.28, 49.08, 26.48, 25.94, 12.62, 12.12. ESI-MS (positive mode) calculated for C₃₄H₃₃N₄ClO₂: m/z 565.11, found: m/z 565.2413.

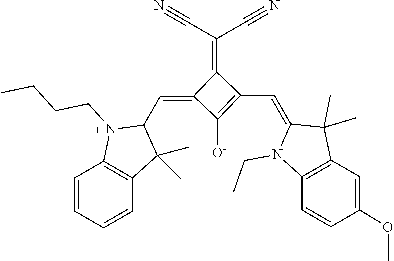 [SMILES: CCCCN1c2ccccc2C(C)(C)C1/C=C1C([O-])=C(/C=C2\N(CC)c3ccc(OC)cc3C2(C)C)C\1=C(C#N)C#N]

(Z)-4-((1-butyl-3,3-dimethyl-3H-indol-1-ium-2-yl)methylene)-3-(dicyanomethylene)-2-(((Z)-1-ethyl-5-methoxy-3,3-dimethylindolin-2-ylidene) methyl) cyclobut-1-en-1-olate (21). It was obtained in 32% yield. ¹H NMR (400 MHZ, DMSO) δ 7.530 (d, J=7.2 Hz, 1H), 7.373 (m, 3H), 7.221 (m, 2H), 6.994 (d, J=9.2 Hz, 1H), 6.296 (s, 1H), 6.248 (s, 1H), 4.090 (m, 2H), 3.994 (m, 2H), 3.815 (s, 3H), 1.677 (s, 12H), 1.392 (m, 2H), 1.284 (t, J=14 Hz, 3H), 0.912 (t, J=14.4 Hz, 3H); ¹³C NMR (100 MHz, DMSO) δ 173.07, 171.67, 170.39, 166.54, 164.90, 163.24, 158.43, 144.49, 142.27, 142.09, 134.99, 128.55, 124.61, 122.68, 119.01, 118.97, 113.80, 112.52, 111.10, 109.54, 88.36, 87.74, 56.32, 50.06, 49.13, 43.87, 29.31, 26.62, 26.09, 19.90, 14.26, 12.54. ESI-MS (positive mode) calculated for C₃₆H₃₈N₄O₂: m/z 558.73, found: m/z 559.3112

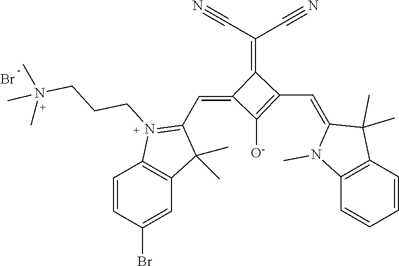

(Z)-4-((5-bromo-3,3-dimethyl-1-(3-(trimethylammonio) propyl)-3H-indol-1-ium-2-yl)methylene)-3-(dicyanomethylene)-2-(((Z)-1,3,3-trimethylindolin-2-ylidene) methyl) cyclobut-1-en-1-olate bromide (22). It was obtained in 52% yield. ¹H NMR (400 MHZ, DMSO) δ 7.818 (s, 1H), 7.615 (m, 2H), 7.577 (m, 1H), 7.474 (m, 1H), 7.406 (m, 1H), 7.334 (m, 1H), 6.388 (s, 1H), 6.185 (s, 1H), 3.973 (m, 2H), 3.672 (s, 3H), 3.158 (s, 9H), 2.132 (m, 1H), 1.689 (s, 12H), 1.277 (m, 6H); ¹³C NMR (100 MHz, CDCl₃) δ 174.81, 173.00, 167.03, 163.22, 144.17, 142.65, 141.46, 131.23, 128.77, 126.22, 122.80, 119.44, 112.71, 89.69, 88.01, 62.88, 53.12, 50.18, 49.18, 46.17, 32.40, 26.57, 20.76, 9.07. ESI-MS (positive mode) calculated for C₃₆H₃₉N₅Br₂O: m/z 717.55, found: m/z 636.2402.

### General Synthesis of Asymmetric Squaric Acid Fluorophores OCTL 12-16 (See FIG. 3)

Individual 3H-indolium salts 6-10 (1.0 mol eq), 11 (1.0 mol eq), and quinoline (5.0 mol eq) were heated under reflux in a 1:1 benzene/n-butanol solvent mixture (40 mL) for 12 h using Dean-Stark apparatus. The reaction mixture was monitored using thin-layer chromatography and vis/NIR spectrophotometry until reaction completion. The solvent was removed under reduced pressure, and the residue was precipitated using ethyl acetate and washed with acetone. The final fluorophore was crystallized using MeOH/ethyl acetate (1:10) and collected using vacuum filtration; the step was repeated to obtain a pure salt in good yield 31-43%.

Mono ((Z)-2-((E)-(3,3-dimethyl-1-(3-(trimethylammonio) propyl) indolin-2-ylidene) methyl)-3-oxo-4-((1,3,3-trimethyl-3H-indol-1-ium-2-yl)methylene) cyclobut-1-enolate mono bromide mono iodide (OCTL 12) Yield 37%; mp>198-200° C. ¹H NMR (400 MHZ, MeOD) δ 1.76-1.77 (m, 12H, CH₃), 2.34 (brs, 2H, CH₂), 3.22 (s, 9H, CH₃), 3.63-3.67 (m, 5H, CH₃&CH₂), 4.23 (brs, 2H, CH₂), 5.89 (s, 1H, CH), 5.99 (s, 1H, CH), 7.21-7.26 (m, 2H, Ar—H), 7.32-7.43 (m, 4H, Ar—H), 7.46-7.50 (m, 2H, Ar—H), ¹³C NMR (100 MHZ, MeOD) & 20.6, 25.6, 26.1, 30.3, 40.0, 49.0, 49.4, 52.5, 52.8, 62.3, 63.3, 109.8, 110.3, 121.9, 122.0, 123.9, 124.6, 128.0, 129.2, 130.0, 135.2, 141.3, 141.8, 142.6, 144.6, 147.5, 182.7. HRMS (m/z) for C₃₃H₄₀N₃O₂⁺, calculated 510.8955, found 510.8995.

(E)-3,3-dimethyl-2-(((Z)-2-oxido-4-oxo-3-((1,3,3-trimethyl-3H-indol-1-ium-2-yl)methylene)-cyclobut-1-en-1-yl)methylene)-1-(3-(trimethylammonio) propyl) indoline-5-sulfonate, mono bromide mono iodide (OCTL 13) Yield 30%. MP 239-241° C.; ¹H NMR (400 MHZ, MeOD) δ 1.73-1.76 (m, 12H, CH₃), 2.33 (brs, 2H, CH₂), 3.23 (s, 9H, CH₃), 3.66-3.72 (m, 5H, CH₂CH₃), 4.16 (brs, 2H, CH₂), 5.87 (s, 1H, CH), 6.03 (s, 1H, CH), 7.28-7.30 (m, 2H, Ar—H), 7.35-7.37 (m, 1H, Ar—H), 7.40-7.42 (m, 1H, Ar—H), 7.50-7.51 (m, 1H, Ar—H), 7.65-7.67 (m, 1H, Ar—H), 7.83 (s, 1H, Ar—H); ¹³C NMR (100 MHZ, MeOD) δ 20.5, 21.5, 25.5, 26.3, 30.6, 40.1, 48.5, 49.7, 52.7, 63.5, 85.9, 86.7, 108.5, 110.7, 119.9, 121.9, 125.0, 126.3, 128.0, 141.05, 142.0, 142.6, 143.7, 168.2, 173.9, 174.2, 179.0, 183.0; HRMS for C₃₃H₃₉N₃O₅S: calculated 589.2610, found 589.6326.

Mono ((Z)-2-((E)-(5-fluoro-3,3-dimethyl-1-(3-(trimethylammonio) propyl) indolin-2-ylidene) methyl)-3-oxo-4-((1,3,3-trimethyl-3H-indol-1-ium-2-yl)methylene) cyclobut-1-enolate) mono bromide monoiodide (OCTL 14) Yield 43%; ¹H NMR (400 MHZ, MeOD) δ 1.76 (s, 12H, CH₃), 2.32 (s, 2H, CH₂), 3.22 (s, 9H, CH₃), 3.62-3.70 (m, 5H, CH₃ & CH₂), 4.20 (s, 2H, CH₂), 5.85 (s, 1H, CH), 5.99 (s, 1H, CH), 7.13 (t, J=8.0 Hz, 1H, ArH), 7.25-7.29 (m, 2H, Ar—H), 7.32-7.36 (m, 2H, Ar—H), 7.40-7.43 (m, 1H, Ar—H), 7.50 (t, J=7.6 Hz, 1H, ArH); ¹³C NMR (100 MHz, MeOD) δ 20.5, 25.5, 26.1, 30.3, 40.1, 52.5, 63.5, 85.3, 86.2, 109.8, 110.1, 110.2, 110.3, 114.0, 114.3, 121.8, 124.7, 127.9, 138.2, 141.9, 142.7, 169.0, 173.4, 173.9, 177.8, 183.0. HRMS (m/z) for C₃₃H₃₉FN₃O₂⁺, calculated 528.3021, found 528.2086.

Mono ((Z)-2-((E)-(5-chloro-3,3-dimethyl-1-(3-(trimethylammonio) propyl) indolin-2-ylidene) methyl)-3-oxo-4-((1,3,3-trimethyl-3H-indol-1-ium-2-yl)methylene) cyclobut-1-enolate) mono bromide monoiodide (OCTL 15) Yield 31%; ¹H NMR (400 MHZ, MeOD) δ 1.75 (s, 12H, CH₃), 2.30-2.31 (m, 2H, CH₂), 3.23 (s, 9H, CH₃), 3.63-3.65 (m, 2H, CH₂), 3.71 (s, 3H, CH₃), 4.17-4.21 (m, 2H, CH₂), 5.84 (s, 1H, CH), 6.01 (s, 1H, CH), 7.26-7.36 (m, 4H, ArH), 7.40-7.42 (m, 1H, Ar—H), 7.44-7.46 (m, 1H, Ar—H), 7.49-7.51 (m, 1H, Ar—H); ¹³C NMR (100 MHZ, MeOD) δ 20.4, 25.5, 26.2, 30.4, 40.0, 49.7, 52.5, 63.5, 85.5, 86.6, 110.5, 121.9, 122.5, 124.9, 127.8, 128.0, 128.8, 141.0, 142.0, 142.6, 143.1, 168.0, 173.9, 183.0. HRMS (m/z) for C₃₃H₃₉ClN₃O₂⁻, calculated 543.1150, found 543.1108.

Mono ((Z)-2-((E)-(5-bromo-3,3-dimethyl-1-(3-(trimethylammonio) propyl) indolin-2-ylidene) methyl)-3-oxo-4-((1,3,3-trimethyl-3H-indol-1-ium-2-yl)methylene) cyclobut-1-enolate) mono bromide monoiodide (OCTL 16) Yield 36%; mp 204-206° C. ¹H NMR (400 MHZ, MeOD) δ 1.75-1.76 (m, 12H, CH₃), 2.31 (s, 2H, CH₂), 3.23 (s, 9H, CH₃), 3.71-3.72 (m, 5H, CH₃ & CH₂), 4.17 (s, 2H, CH₂), 5.84 (s, 1H, CH), 6.01 (s, 1H, CH), 7.28-7.32 (m, 2H, Ar—H), 7.35-7.37 (m, 1H, Ar—H), 7.40-7.43 (m, 1H, Ar—H), 7.49-7.51 (m, 2H, Ar—H), 7.58-7.61 (m, 1H, Ar—H); ¹³C NMR (100 MHZ, MeOD) δ 20.4, 25.5, 26.1, 30.4, 39.9, 49.6, 52.5, 63.3, 85.5, 86.6, 110.6, 111.1, 116.0, 121.9, 124.9, 125.4, 128.0, 130.8, 141.4, 142.0, 142.5, 173.6, 178.2, 182.9. HRMS (m/z) for C₃₃H₃₉BrN₃O₂⁺, calculated 588.6220, found 588.6233.

### Example 2-Structure Inherent Targeting

Squaraine fluorophores of example 1 display ultrabright optical properties and optimal pharmacokinetics, allowing high contrast and durable near-infrared imaging for fluorescence-guided surgery of, e.g., ovarian cancer. The primary mechanisms of the tumor targetability of squaraines involve its rapid diffusion across tumor vasculature and cellular uptake via organic cation transporters (OCTs) and retention in the lysosome. These features are optimal for rapid fluorescence recovery, durable retention, and excellent targetability upon intravenous administration, allowing for intraoperative imaging for a sufficiently long time and the detection of small peritoneal dissemination of, e.g., ovarian cancer.

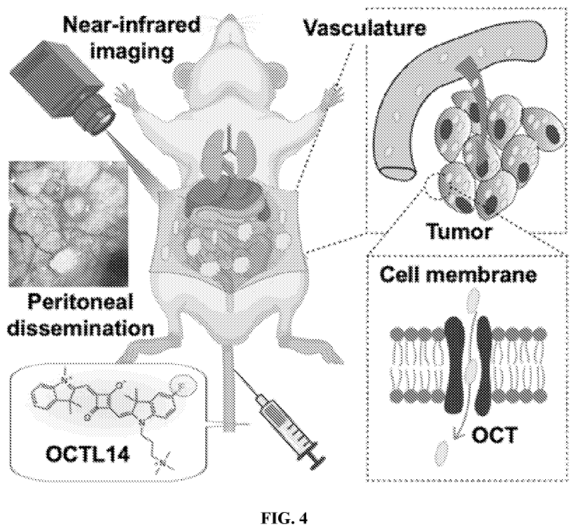

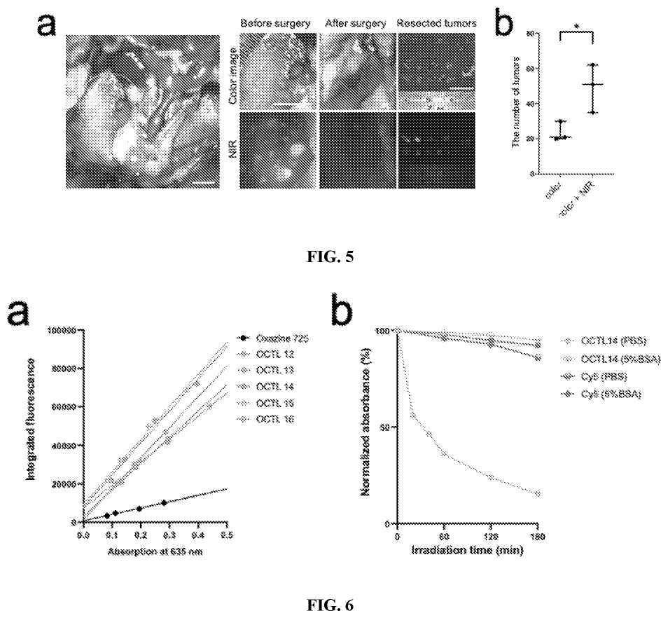

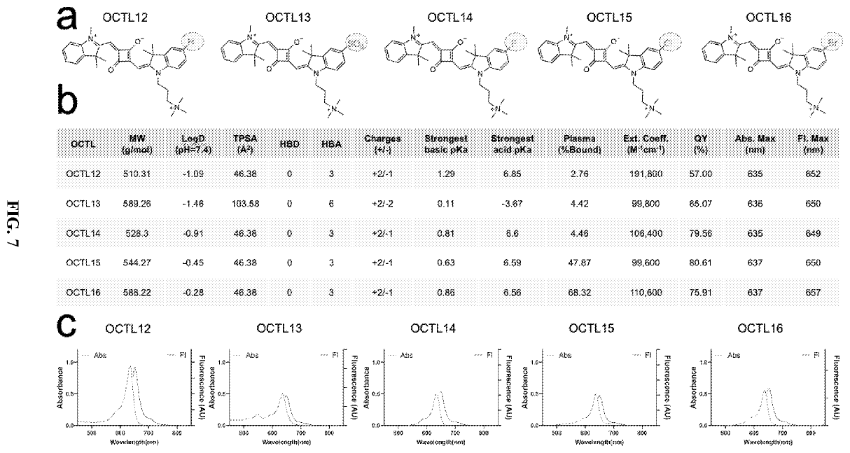

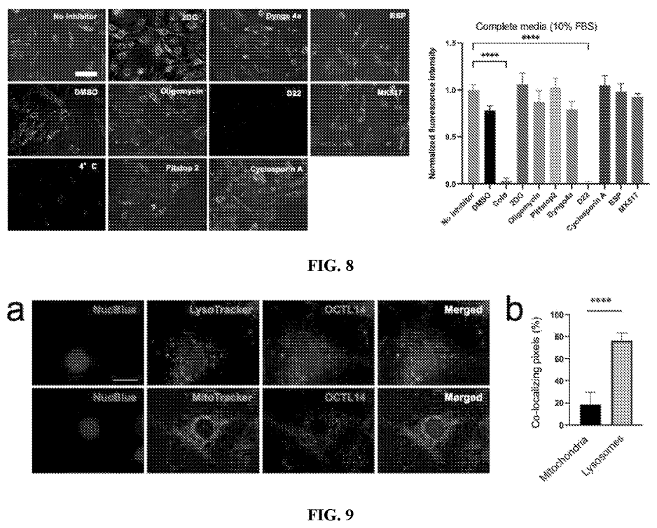

Structure-inherent targeting: Squaraines are selectively taken up by cancer cells via organic cation transporters (OCTs), overexpressed by cancer cells (FIG. 4). This high affinity is mediated by the unique structure of the molecule. This was achieved this by introducing an asymmetric molecular conformation and surface charges. In this way, squaraines can be up-taken rapidly via transporter and selectively accumulate in cancer cells, not normal cells. In addition, we have shown that once they are taken up by cancer cells, squaraines can stay in lysosomes for hours for durable imaging. This feature is critical to have a clear contrast between cancerous and normal tissue for clear cancer imaging during surgery or medical procedures. An example of a squaraine, OCTL14, provided precise localization of cancer tissues 6 h post-injection and enabled the surgeon to achieve complete resection of tumors, including a small nodule measuring <1 mm to 5 mm in size with no residual tumor (See FIG. 5).

Serum stability: Historically, squaraines showed instability owing to their highly chemically- and photolytically-labile oxocyclobutenolate ring, significantly limiting their applications. A robust solution to the stability issues was developed by synthesizing an intramolecular salt bridge to protect the central oxocyclobutenolate ring of the squaraine fluorophore with a quaternary ammonium cation which increased the molar absorptivity and quantum yield in serum due to the enhanced rigidity associated with its locked structure. The resulting squaraines are ultrabright and stable in serum (See FIG. 6).

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (II): 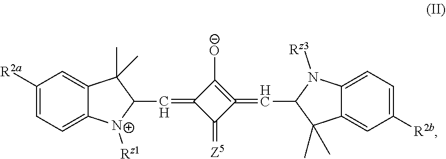
or a pharmaceutically acceptable salt thereof, wherein:
Z⁵ is O, S, NR^{a}, or CR^{a}R^{b};
R^{a} is F, Cl, Br, I, NO₂, CN, R^{a1}, OR^{a1}, SR^{a1}, N(R^{a1})₂, SO₂R^{a1}, SO₂N(R^{a1})₂, C(O)R^{a1}, C(O)OR^{a1}, OC(O)R^{a1}, C(O)N(R^{a1})₂, N(R^{a1}) C(O)R^{a1}, OC(O)N(R^{a1})₂, or N(R^{a1}) C(O)N(R^{a1})₂, wherein each R^{a1} is independently selected from hydrogen, C₁₋₈ alkyl, aryl, C₁₋₈ heteroaryl, C₃₋₈ cycloalkyl, and C₁₋₈ heterocyclyl; and
R^{b} is F, Cl, Br, I, NO₂, CN, R^{b1}, OR^{b1}, SR^{b1}, N(R^{b1})₂, SO₂R^{b1}, SO₂N(R^{b1})₂, C(O)R^{b1}, C(O)OR^{b1}, OC(O)R^{b1}, C(O)N(R^{b1})₂, N(R^{b1}) C(O)R^{b1}, OC(O)N(R^{b1})₂, or N(R^{b1}) C(O)N(R^{b1})₂, wherein each R^{b1} is independently selected from hydrogen, C₁₋₈ alkyl, aryl, C₁₋₈ heteroaryl, C₃₋₈ cycloalkyl, and C₁₋₈ heterocyclyl;
or R^{a} and R^{b} together form a ring;
R^{2a} is selected from F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}; C(O)N(R^{2a*})₂, N(R^{2a*}) C(O)R^{2a*}, OC(O)N(R^{2a*})₂, and N(R^{2a*})C(O)N(R^{2a*})₂, wherein R^{2a*} is in each case independently selected from hydrogen, C₁₋₈ alkyl, aryl, C₁₋₈ heteroaryl, C_{3.8} cycloalkyl, and C₁₋₈ heterocyclyl;
R^{2b} is selected from F, Cl, Br, I, NO₂, CN, R^{2b*}, OR^{2b*}, SR^{2b*}, N(R^{2b*})₂, SO₃R^{2b*}, SO₂R^{2b*} SO₂N(R^{2b*})₂, C(O)R^{2b*}; C(O)OR^{2b*}, OC(O)R^{2b*}; C(O)N(R^{2b*})₂, N(R^{2b*}) C(O)R^{2b*}, OC(O)N(R^{2b*})₂, N(R^{2b*}) C(O)N(R^{2b*})₂, wherein R^{2b*} is in each case independently selected from hydrogen, C₁₋₈ alkyl, aryl, C₁₋₈heteroaryl, C₃₋₈cycloalkyl, or C₁₋₈heterocyclyl;
provided that at least one of R^{2a} and R^{2b} is other than hydrogen;
R^{z1} is C₁₋₈ alkyl, optionally substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H; and
R^{z3} is C₁₋₈ alkyl, optionally substituted by aryl, heteroaryl, F, Cl, Br, I, CN, C₁₋₃ alkyl, C₁₋₃ haloalkyl, OH, OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H;
provided that the compound is not any of the following compounds:

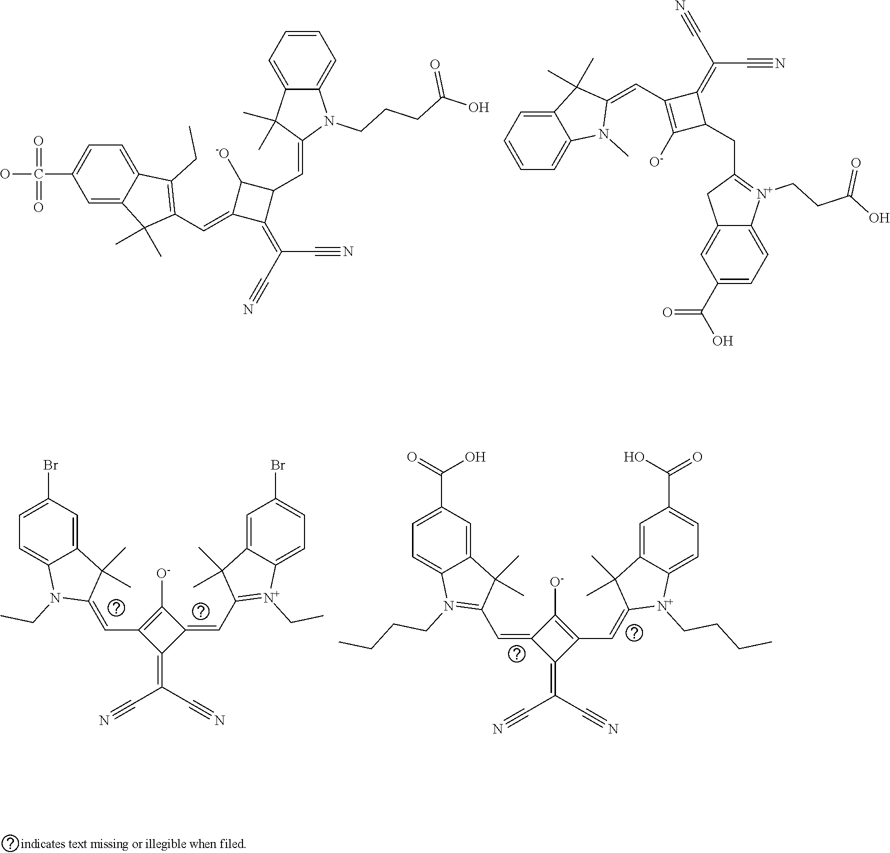

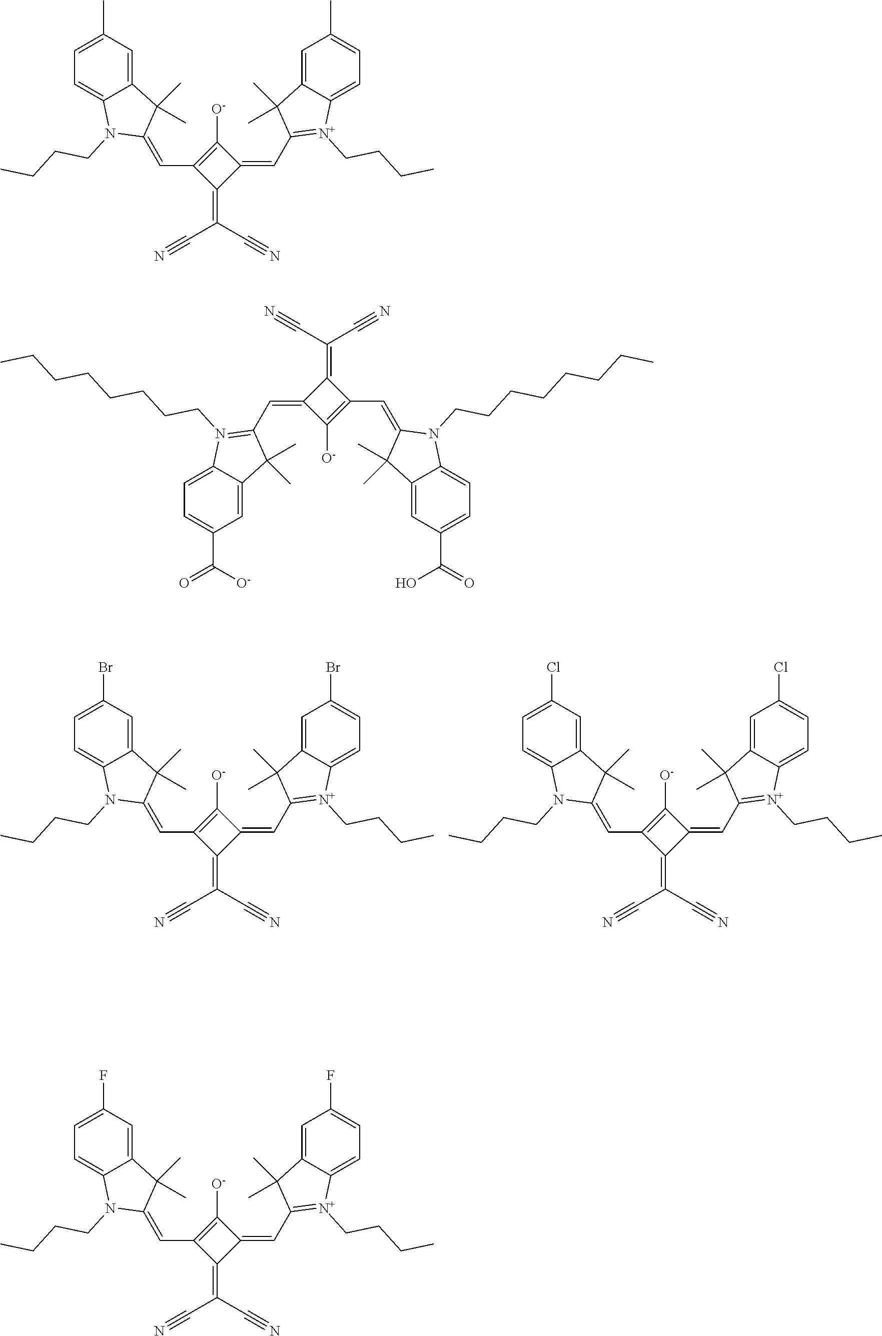

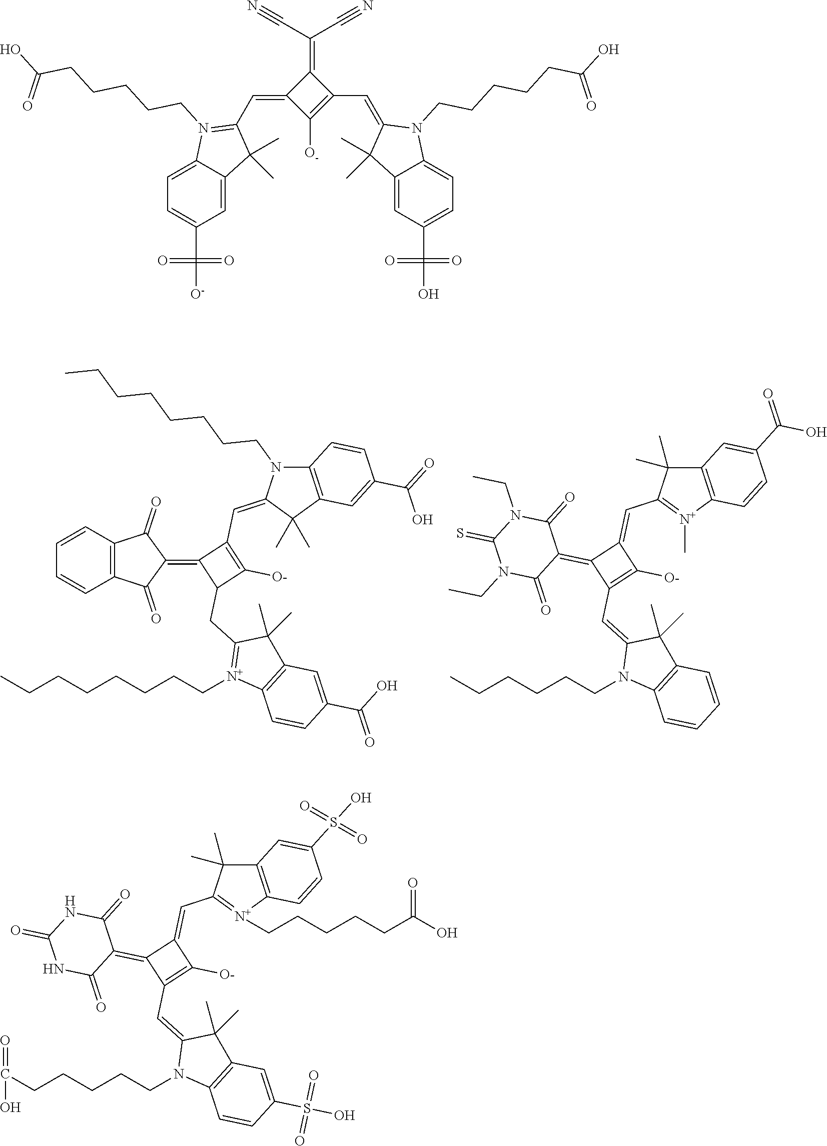

2. The compound of claim 1, wherein Z⁵ is CR^{a}R^{b}.

3. The compound of claim 1, wherein R^{a} and R^{b} are each independently selected from CN, F, Cl, CF₃, CHF₂, CHF₂, CO₂H, CONH₂, CO₂C₁₋₃alkyl, COC₁₋₃alkyl, CONHC₁₋₃alkyl, CO₂C₁₋₃haloalkyl, COC₁₋₃haloalkyl, CONHC₁₋₃ haloalkyl, CO₂C₁₋₃alkyl-OH, COC₁₋₃alkyl-OH, and CONHC₁₋₃alkyl-OH.

4. The compound of claim 1, wherein R^{a} and R^{b} together form a ring having the formula: 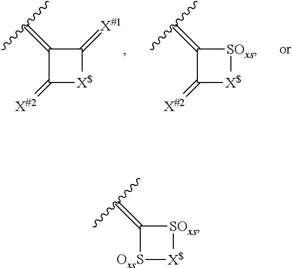
wherein each xs is independently 1 or 2;
X^{#1} and X^{#2} are each independently selected from oxo, thioxo, imino, and CH₂, provided that both X^{#1} and X^{#2} are not each CH₂;
X^{$} is -ethylene-, -propylene-, -ethenyl-, O-methylene, —NH-methylene-, O-ethylene, —NH-ethylene-, —O-ethylene-O—, —NH-ethylene-O—, —NH-ethylene-NH—, —O-propylene-, —NH-propylene-, —O-propylene-O—, —NH-propylene-O—, —NH-propylene-NH—, aryl, cycloalkyl, heteroaryl, or heterocyclyl,
wherein said methylene, ethylene, ethenyl, propylene, propenyl, aryl, cycloalkyl, heteroaryl, and heterocyclyl may be substituted with 1, 2, or 3 substituents independently selected from F, Cl, Br, I, CN, NH₂, COOH, CONH₂, —(CH₂CH₂O)ₒCH₃ (o=1-20), —(CH₂CH₂O)ₒH (o=1-20), C₁₋₃alkyl, C₁₋₃haloalkyl, C₁₋₃alkoxy, C₁₋₃haloalkoxy, C₁₋₃aminoalkyl, and C₁₋₃aminohaloalkyl.

5. The compound of claim 1, wherein R^{a} and R^{b} together form a ring having the formula: 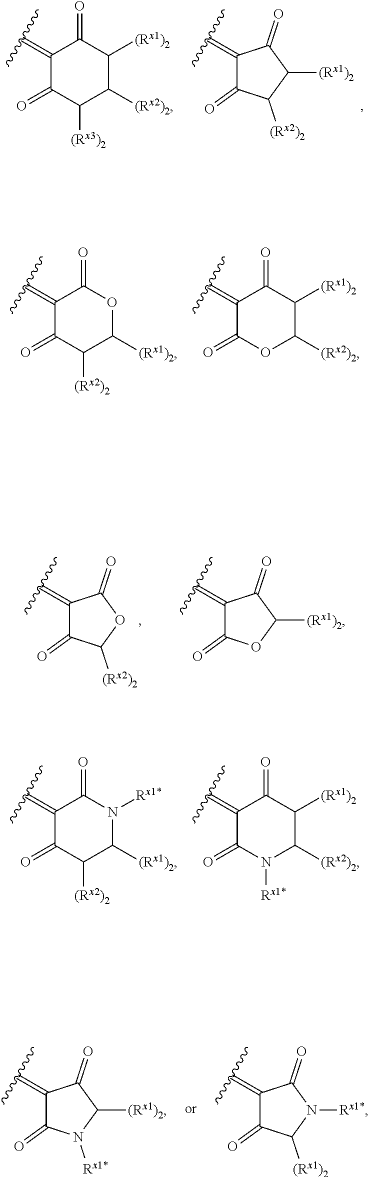
wherein
each R^{x1} is independently selected from H, F, Cl, Br, I, CN, NH₂, COOH, CONH₂, —(CH₂CH₂O)ₒCH₃ (o=1-20), —(CH₂CH₂O)ₒH (o=1-20), C₁₋₃alkyl, C₁₋₃haloalkyl, C₁₋₃alkoxy, C₁₋₃haloalkoxy, C₁₋₃aminoalkyl, and C₁₋₃aminohaloalkyl; and
each R^{x1*} is independently selected from H, CONH₂, —(CH₂CH₂O)ₒCH₃ (o=1-20), —(CH₂CH₂O)ₒH (o=1-20), C₁₋₃alkyl, and C₁₋₃haloalkyl, or
two geminal R^{x1} groups can together form an oxo, two geminal or adjacent R^{x1} groups can together form a ring, or two adjacent R^{x1} groups can together form a double bond.

6. The compound of claim 1, wherein the compound has Formula (I): 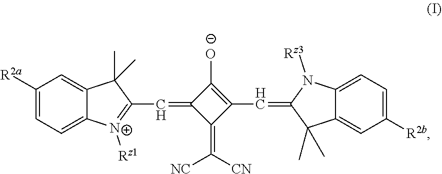
or a pharmaceutically acceptable salt thereof.

7. The compound of claim 1, wherein R^{2a} is selected from H, Cl, Br, CN, C₁₋₆ alkoxy, NH₂, NH(C₁₋₆ alkyl), N(C₁₋₈ alkyl)₂, and C(O)OH.

8. The compound of claim 1, wherein R^{2a} is selected from Cl, Br, C₁₋₆ alkoxy, and C(O)OH.

9. The compound of claim 1, wherein R^{2b} is selected from H, Cl, Br, CN, C₁₋₆ alkoxy, NH₂, NH(C₁₋₆ alkyl), N(C₁₋₈ alkyl)₂, and C(O)OH.

10. The compound of claim 1, wherein R^{2b} is selected from Cl, Br, C₁₋₆ alkoxy, and C(O)OH.

11. The compound of claim 1, wherein:
R^{2a} is selected from Cl, Br, CN, C₁₋₆ alkoxy, NH₂, NH(C₁₋₆ alkyl), N(C₁₋₈ alkyl)₂, and C(O)OH; and
R^{2b} is selected from Cl, Br, CN, C₁₋₆ alkoxy, NH₂, NH(C₁₋₆ alkyl), N(C₁₋₈ alkyl)₂, and C(O)OH.

12. The compound of claim 1, wherein
R^{2a} is selected from C₁₋₆ alkoxy; and
R^{2b} is selected from C₁₋₆ alkoxy and Cl.

13. The compound of claim 1, wherein:
R^{2a} is hydrogen; and
R^{2b} is selected from Cl, Br, CN, C₁₋₆ alkoxy, NH₂, NH(C₁₋₆ alkyl), N(C₁₋₈ alkyl)₂, and C(O)OH.

14. (canceled)

15. The compound of claim 1, wherein R^{z1} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H.

16. The compound of claim 1, wherein R^{z3} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H.

17. The compound of claim 1, wherein:
R^{z1} is C₁₋₈ alkyl, optionally substituted by OC₁₋₃ alkyl, NH₂, NHC₁₋₃ alkyl, N(C₁₋₃ alkyl)₂, N⁺(C₁₋₃ alkyl)₃, SO₃H, PO₃H₂, or CO₂H;
R^{z3} is C₁₋₈ alkyl, substituted by NH₂, NHC₁₋₃ alkyl, N(C₁₋₃ alkyl)₂, N⁺(C₁₋₃ alkyl)₃, SO₃H, or PO₃H₂.

18. (canceled)

19. The compound of claim 1, selected from any one of the following compounds: 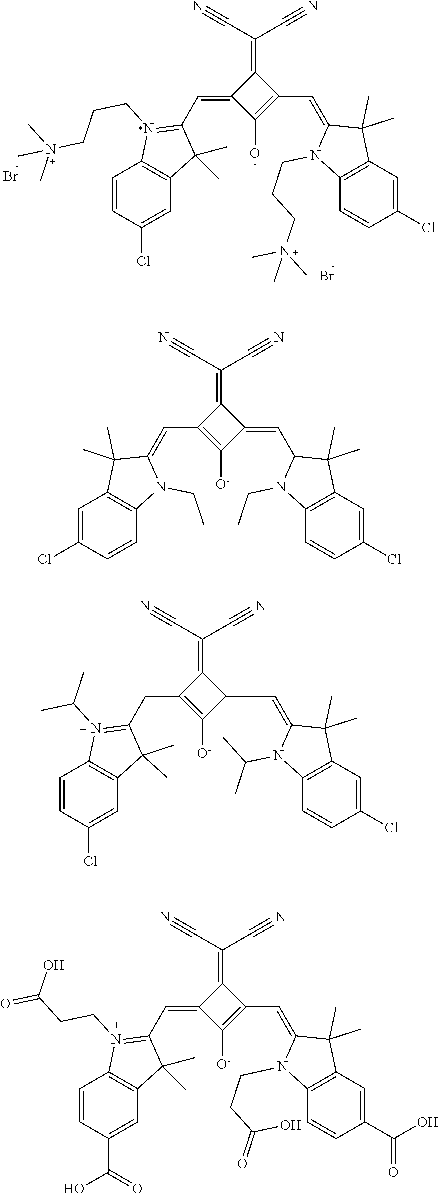 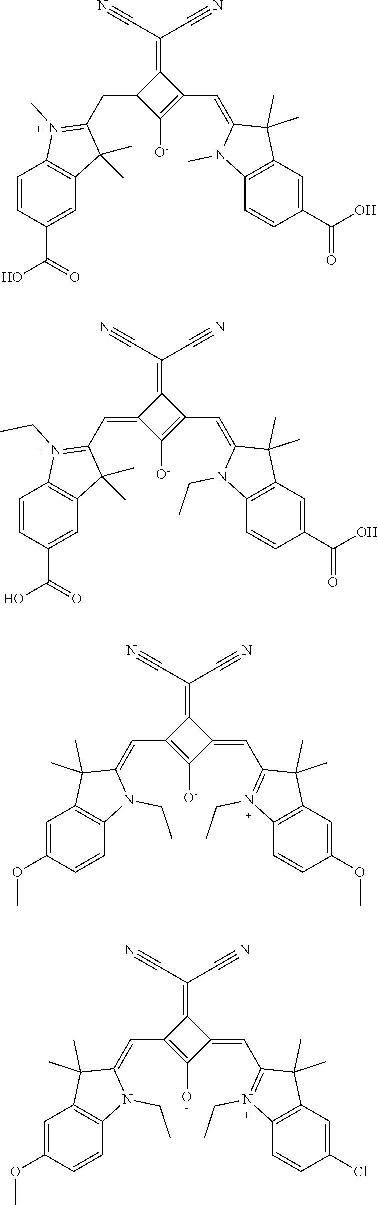 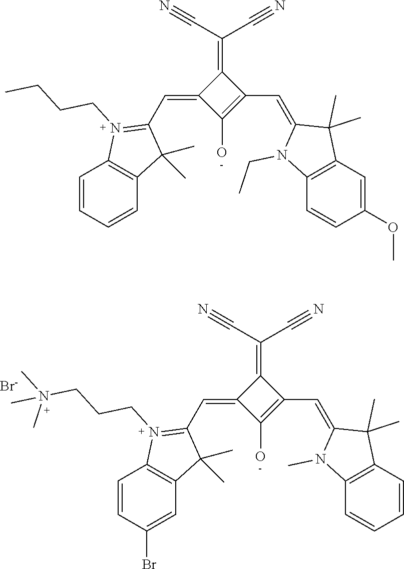
or a pharmaceutically acceptable salt thereof.

20. A pharmaceutical composition comprising a compound of claim 1, or a pharmaceutically acceptable salt thereof, and a pharmaceutically acceptable carrier.

21. A method of imaging a cancerous tumor in a subject, the method comprising:
i) administering to the subject an effective amount of a compound of claim 1, or a pharmaceutically acceptable salt thereof;
ii) waiting a time sufficient to allow the compound to accumulate in the cancerous tumor to be imaged; and
iii) imaging the cancerous tumor with a fluorescence imaging technique.

22-24. (canceled)

25. A method of treating cancer, the method comprising:
i) imaging a cancerous tumor in a subject according to the method of claim 21; and
ii) surgically removing the cancerous tumor from the subject.
